(19) United States
(12) Patent Application Publication
(10) Pub. No.: US 2016/0051709 A1
(43) Pub. Date: Feb. 25, 2016

(54) COMPOSITION-CONTROLLED NOBLE METAL-TRANSITION METAL SMALL NANOPARTICLE ALLOYS WITH NIR-EMISSION AND HIGH T2 RELAXIVITY AND METHOD FOR MAKING SAME

(71) Applicants: Jill Erin Millstone, Pittsburgh, PA (US); Lauren E. Marbella, Pittsburgh, PA (US); Christopher Michael Andolina, Pittsburgh, PA (US)

(72) Inventors: Jill Erin Millstone, Pittsburgh, PA (US); Lauren E. Marbella, Pittsburgh, PA (US); Christopher Michael Andolina, Pittsburgh, PA (US)

(73) Assignee: University of Pittsburgh - Of The Commonwealth System of Higher Education

(21) Appl. No.: 14/804,982

(22) Filed: Jul. 21, 2015

Related U.S. Application Data
(60) Provisional application No. 62/027,172, filed on Jul. 21, 2014.

(51) Int. Cl. A61K 49/18; A61K 49/12

(52) U.S. Cl. CPC A61K 49/1824; A61K 49/126

(57) ABSTRACT

A method for producing small nanoparticles of a discrete noble metal-transition metal nanoparticle alloy, comprising: mixing, at room temperature in air, a first aqueous solution having a first molar ratio of a noble metal and a transition metal with an organic ligand and a reducing agent. A method for producing small nanoparticles of a discrete gold-cobalt nanoparticle alloy, comprising: mixing, at room temperature in air, a first aqueous solution having a first molar ratio of HAuCl₄ and Co(NO₃)₂ with an organic ligand comprising poly(ethylene glycol) methyl ether thiol (PEGSH, average Mₙ=1000 Da) and a reducing agent comprising sodium borohydride (NaBH₄).

### CROSS REFERENCE TO RELATED APPLICATIONS

This application claims the benefit of the filing date of U.S. provisional patent application Ser. No. 62/027,172, filed on Jul. 21, 2014, the entirety of which is incorporated herein by reference for all purposes.

### STATEMENT REGARDING FEDERALLY SPONSORED RESEARCH OR DEVELOPMENT

This invention was made with government support under grant #1253143 from the National Science Foundation. The government has certain rights in the invention.

### BACKGROUND OF THE DISCLOSURE

The present disclosure relates to the development of small, colloidal magnetic nanoparticles: multimodal material platforms. Nanoparticle alloys are a class of materials that allow the implementation and in some cases, enhancement of multiple functionalities into a single structure. Yet, optimization of multimodalities has not been fully explored due to limitations achieving tunability across all composition space for two metals that possess differing, favorable properties, but also large immiscibility. We present a synthetic method that allows composition-tunability for small (d=2-3 nm), discrete noble metal-transition metal nanoparticle alloys, specifically gold-cobalt alloys (% Co=0-100%), fully characterized by optical spectroscopy, high-resolution transmission electron microscopy (HRTEM), inductively-coupled plasma mass spectrometry (ICP-MS), X-ray photoelectron spectroscopy (XPS), photoluminescence, and proton nuclear magnetic resonance (¹H NMR) techniques. In particular, ¹H NMR methods allow the simultaneous evaluation of composition-tunable magnetic properties as well as molecular characterization of the colloid. The method herein provides the means to optimize bimodal imaging capabilities of AuₓCo_{y}NPs that exhibit near infrared (NIR) photoluminescence (PL), as well as favorable T₂ relaxivities. By altering the nanoalloy elemental composition, we found the optimal composition, Co₈₀Au₂₀NP (initial molar ratio Co:Au=80:20) that exhibits exceptional brightness and high per-particle relaxivity for use as a dual NIR-T₂-weighted contrast imaging agent.

Noble metal nanoparticle (NP) properties strongly depend on particle size, shape, surface chemistry, and composition. Of these, changing the composition to incorporate more than one metal into a single particle presents the opportunity to incorporate multiple functionalities into a single architecture and amplify interesting physical and chemical properties. A variety of bimetallic nanostructures exist; Janus and core-shell nanostructures provide a straightforward architecture to combine individual properties, while solid-solution nanoalloys offer the opportunity to powerfully unite and enhance properties through synergistic effects. However, alterations to composition often necessitate changes to size, shape, or surface chemistry. In order to evaluate and optimize multimodal properties, all synthetic handles must be accessible independent of one another. However, composition is a particularly difficult parameter to isolate and tune due to the limitations associated with forming alloys across a range of compositions, according to classical Hume-Rothery rules, in particular while holding the total size of the particle constant.

Recently we reported the NIR PL properties of small diameter (d=2-3 nm) gold and gold-copper NP alloys. The NIR PL is believed to be the result of Au(I)-thiolate bonds on the surface of the particle. The Au and AuₓCu_{y}NP alloys showed high brightness (brightness=molar extinction coefficient (ε)×quantum yield (Φ)), making these materials well-suited for NIR imaging and molecular tracking. These superior imaging capabilities can be extended by alloying gold with a ferromagnetic material (Ni, Co, or Fe). It is well-known that when reduced to the nanoscale, ferromagnetic materials begin to exhibit superparamagnetism that can be used in applications ranging from data storage to theranostics. The utility of these properties have been thoroughly investigated for magnetic resonance imaging (MRI), mainly in the form of negative T₂ contrast applications.

Noble metal and magnetic metal bimetallic nanostructures have been synthesized in the past, but the description of optical properties is lacking. Where these descriptions are present, the optical effect described is that of a localized surface plasmon resonance (LSPR) which is a collective response of the conduction electrons at the surface of the material and is characteristic of larger noble metal nanostructures (dimensions >10 nm). In essentially all cases of noble metal-magnetic metal hybrids, a severe dampening and of the LSPR is observed. However, in certain cases, the remaining localized surface plasmon resonance from gold has been shown to enhance the magneto-optical effect of maghemite and cobalt. Recent studies show that the unique combination of gold and cobalt is a promising multimodal imaging agent, and a cobalt-gold core-shell-like nanowonton structure has been shown to possess high per-particle relaxivity reported compared to other commercial available agents. Since we have demonstrated that Au-transition metal nanoparticle alloys in the biologically compatible size regime (d=2-3 nm) are among the brightest NIR probes, we hypothesized that by using our surface chemistry-directed approach, we could isolate an array of discrete gold-cobalt (AuₓCo_{y}) nanoparticle alloy compositions at this size that exhibit tunable magnetic susceptibility while maintaining NIR luminescence. By alloying gold and cobalt on the nanoscale, in the same size range, we have the tools to produce bimodal imaging agents that exhibit remarkable brightness and per-particle relaxivity for use as dual NIR-T₂ contrast imaging agents that can be tuned by varying the Au:Co ratio. Although an intermetallic and core-shell structures have been reported, forming a solid solution of gold and cobalt while maintaining a constant diameter is synthetically challenging due to the bulk-immiscibility of the two metals across all composition space.

Despite these difficulties, we report the first discrete, small diameter (d=2-3 nm) compositionally tunable AuₓCo_{y}NP alloys that exhibit tunable magnetic susceptibility as well as superior NIR and T₂-relaxivity properties compared to NIR-emitting lanthanide complexes and superparmagnetic iron oxide nanoparticles (SPIONs), respectively. Our low-temperature, aqueous synthetic approach allows for tunability across a range of AuₓCo_{y}NP compositions (0 to 100% Co). This environmentally-friendly method provides the framework to evaluate the optimal NIR brightness and per-particle relaxivity through composition manipulation, and can be extended to other multimodal systems of various compositions in this size regime (d=2-3 nm). This work emphasizes the significance of composition-tunable alloys in optimizing bimodal capabilities.

### SUMMARY

In a preferred aspect, the present disclosure comprises a method for producing small nanoparticles of a discrete gold-cobalt nanoparticle alloy, comprising: mixing, at room temperature in air, a first aqueous solution having a first molar ratio of HAuCl₄ and Co(NO₃)₂ with an organic ligand comprising poly(ethylene glycol) methyl ether thiol (PEGSH, average Mₙ=1000 Da) and a reducing agent comprising sodium borohydride (NaBH₄).

In another preferred aspect, the method of the present disclosure further comprises characterizing the gold-cobalt nanoparticles of the first aqueous solution by photoluminescence, by other property of the gold-cobalt alloy nanoparticles from the first aqueous solution and/or by one or more of UV-visible spectroscopy, inductively coupled plasma mass spectrometry (ICP-MS), X-ray photoelectron spectroscopy (XPS), PL, HRTEM techniques, and ¹H nuclear magnetic resonance (NMR) techniques.

In yet another preferred aspect, the method of the present disclosure further comprises mixing, at room temperature in air, a second aqueous solution having a second molar ratio of HAuCl₄ and Co(NO₃)₂ with an organic ligand comprising poly(ethylene glycol) methyl ether thiol (PEGSH, average Mₙ=1000 Da) and a reducing agent comprising sodium borohydride (NaBH₄).

In another preferred aspect, the method of the present disclosure further comprises characterizing the gold-cobalt nanoparticles of the second aqueous solution by photoluminescence, by other property of the gold-cobalt alloy nanoparticles from the second aqueous solution and/or by one or more of UV-visible spectroscopy, inductively coupled plasma mass spectrometry (ICP-MS), X-ray photoelectron spectroscopy (XPS), PL, HRTEM techniques, and ¹H nuclear magnetic resonance (NMR) techniques.

In an additional preferred aspect, the method of the present disclosure further comprises comparing the characterization results for the gold-cobalt nanoparticles from the first and second aqueous solutions.

In another preferred aspect, the present disclosure comprises a method for producing small nanoparticles of a discrete noble metal-transition metal nanoparticle alloy, comprising: mixing, at room temperature in air, a first aqueous solution having a first molar ratio of a noble metal and a transition metal with an organic ligand and a reducing agent.

In another preferred aspect, the method of the present disclosure further comprises characterizing the noble metal-transition metal nanoparticles of the first aqueous solution by photoluminescence, by other property of the noble metal-transition metal alloy nanoparticles from the first aqueous solution and/or by one or more of UV-visible spectroscopy, inductively coupled plasma mass spectrometry (ICP-MS), X-ray photoelectron spectroscopy (XPS), PL, HRTEM techniques, and ¹H nuclear magnetic resonance (NMR) techniques.

In yet another preferred aspect, the method of the present disclosure further comprises mixing, at room temperature in air, a second aqueous solution having a second molar ratio of noble metal-transition metal with an organic and a reducing agent.

In another preferred aspect, the method of the present disclosure further comprises characterizing the noble metal-transition metal nanoparticles of the second aqueous solution by photoluminescence, by other property of the noble metal-transition metal alloy nanoparticles from the second aqueous solution and/or by one or more of UV-visible spectroscopy, inductively coupled plasma mass spectrometry (ICP-MS), X-ray photoelectron spectroscopy (XPS), PL, HRTEM techniques, and 1H nuclear magnetic resonance (NMR) techniques.

In yet another preferred aspect, the method of the present disclosure further comprises comparing the characterization results for the noble metal-transition metal nanoparticles from the first and second aqueous solutions.

In another preferred aspect of the method of the present disclosure, the noble metal is selected from the group consisting of gold (Au), silver (Ag) and platinum (Pt) and the transition metal is selected from the group consisting of copper (Cu), cobalt (Co), nickel (Ni), zinc (Zn), ruthenium (Ru), rhodium (Rh), aluminum (Al), iron (Fe) and palladium (Pd).

In another preferred aspect, the present disclosure comprises a dual NIR-T₂-weighted contrast imaging agent comprising nanoparticles of a discrete gold-cobalt nanoparticle alloy having a composition of Co₈₀Au₂₀.

In another preferred aspect of the dual NIR-T₂-weighted contrast imaging agent of the present disclosure, an initial molar % Co=80% and an actual % Co incorporated=52%.

In another preferred aspect, the present disclosure comprises a dual NIR-T₂-weighted contrast imaging agent comprising nanoparticles of a discrete gold-cobalt nanoparticle alloy having a composition of Co₅₀Au₅₀.

In another preferred aspect of the dual NIR-T₂-weighted contrast imaging agent of the present disclosure, an initial molar % Co=15% and an actual % Co incorporated=62%.

In another preferred aspect of the dual NIR-T₂-weighted contrast imaging agent of the present disclosure, the gold-cobalt nanoparticles have a diameter ranging from about 2 nm to about 3 nm.

In another preferred aspect of the dual NIR-T₂-weighted contrast imaging agent of the present disclosure, the gold-cobalt nanoparticles are capped with a biologically compatible capping ligand.

### BRIEF DESCRIPTION OF THE DRAWINGS

FIG. 1 A) HRTEM micrograph of AuxCo [symbol] yNPs with an initial molar ratio of 60% Co. B) close-up of an individual AuxCoyNP lattice and C) the corresponding indexed FFT.

FIG. 2 shows magnetic susceptibility of AuₓCo_{y}NPs increases as % Co increases. Error bars in both χ and % Co incorporated represent the standard error across 5 separate experiments.

FIG. 3 shows the optimal composition for bimodal NIR-T2 weighted MRI imaging occurs at Co80Au20PEGSH (actual % Co incorporation ˜50%).

FIG. 4 shows the percent transition metal (Cu or Co) incorporated in the final AuₓTM_{y}NP as a function of percent transition metal initially added in the synthesis evaluated by ICP-MS. Error bars represent the standard error of at least 4 independent measurements for two phase syntheses and 10 independent measurements for one phase syntheses.

FIG. 5 shows (A) ¹H NMR spectra of PEGSH alone in solution and in the presence of HAuCl₄, Cu(NO₃)₂, and Co(NO₃)₂. All metal:thiol ratios=1:1 and all solutions were prepared in 90% H₂O 10% D₂O. (B) Diffusion coefficients extracted from monitoring the ¹H resonance of the (O—CH₂—CH₂)ₙ, repeat unit of the precursors in (A).

FIG. 6 shows MALDI-TOF-MS of high molecular weight metal-thiolate species present in one-phase aqueous (A) Au—Cu and (B) Au—Co nanoparticle syntheses.

FIG. 7 shows A) HRTEM image of AuₓCo_{y}NPs (y=26.8±2.0%) B) magnified image of an individual AuₓCo_{y}NP and C) the corresponding FFT.

FIG. 8 shows the magnetic susceptibility of AuₓCo_{y}NPs increases as % Co increases. Error bars in both χ and % Co incorporated represent the standard error of at least 6 independent experiments.

FIG. 9 shows A) Photoluminescence of AuₓCo_{y}NPs in D₂O showing representative emission spectra. B) Maximum emission wavelength as a function of % Co incorporated.

FIG. 10 shows the optimal AuₓCo_{y}NP composition for bimodal NIR-T₂ contrast imaging occurs at y=48.1±2.7% Co incorporation.

FIG. 11 shows MALDI spectra of PEGSH (Mₙ=1000 Da) as received from A) Laysan Bio, Inc. lot number 134-4 and B) Sigma Aldrich, batch MKBD6808V.

FIG. 12 shows HRTEM micrographs for the following AuₓCo_{y}NP alloy compositions A) y=1.6±0.1%, B) y=7.7±0.7%, C) y=48.1±2.7%, D) y=62.0±2.0%, E) y=80.7±2.5%, and F) y=100±0%, including a wideview, close-up of an individual particle, and the corresponding indexed FFT used to determine average lattice constant.

FIG. 13 shows histograms of AuₓCo_{y}NPs size distributions based on HRTEM micrographs for A) y=1.6±0.1%, B) y=7.7±0.7%, C) y=26.8±2.0%, D) y=48.1±2.7%, E) y=62.0±2.0%, F) y=80.7±2.5%, and G) y=100±0. N represents the number of particles used for size determination; d represents average diameter±the standard deviation of the average.

FIG. 14 shows representative area STEM-HAADF images and corresponding EDS spectra of AuₓCo_{y}NPs on ultra-thin carbon 3-5 nm copper mesh grid using a JEOL JEM 2100F.

FIG. 15 shows survey XPS spectrum of AuₓCo_{y}NPs (y=48.1±2.7%).

FIG. 16 shows high resolution XPS spectra of Au4f and Co2p regions for all NP compositions (top). Plot of binding energy of Au4f_{7/2} (bottom left) and Co2p_{3/2} (bottom right) as a function of % Co and % Au incorporation in the NP, respectively. Exact % Co and % Au incorporations measured by ICP-MS are reported.

FIG. 17 shows high resolution XPS spectra of B1s of H₃BO₃, CoNPs, AuNPs, and AuₓCo_{y}NPs (y=48.1±2.7%). The H₃BO₃ control shows a peak at 194.3 eV. No peaks are observed in the NP B1s spectra, in particular in the shaded region that corresponds to metal-boride species.

FIG. 18 shows percent Co incorporated into the final nanoparticle as a function of the initial molar percent Co added during synthesis (as determined by ICP-MS). The data points represent the experimental data, and the dotted line represents the theoretical composition for 1:1 molar incorporation.

FIG. 19 shows a stack plot of ¹H NMR spectra from AuₓCo_{y}NPs recorded for the Evans' method. The asterisk represents the HDO ¹H NMR peak from pure D₂O in the capillary tube. As % Co increases, the distance between the HDO peaks from solvent inside the capillary vs. solvent inside the colloidal suspension increases. The HDO peak from the colloid also experiences dephasing as % Co increases as a result of T₂ relaxation enhancement line-broadening: FWHM=(πT₂)⁻¹.

FIG. 20 shows a plot of % Co incorporation vs. total magnetic susceptibility, X. Varying the % Co incorporated in the AuₓCo_{y}NP alloy composition allows for tunable magnetic susceptibility. Compositions with high % Co incorporation show higher susceptibility values. Both x and y error bars represent the standard error associated with averaged ICP-MS values for a given initial molar ratio and susceptibility values, respectively, for at least 6 independent trials of each composition.

FIG. 21 shows a ¹H NMR spectral region containing ¹H NMR resonances of the PEGSH capping ligand. The top spectrum shows the ^{1H} NMR of free PEGSH in D₂O and the spectra below show the ¹H NMR of PEGSH-capped AuₓCo_{y}NPs. No free PEGSH is detected in the particle-bound spectra, as indicated by the red dotted line, highlighting the absence of peak 1.

FIG. 22 shows a normalized UV-vis-NIR spectra of AuₓCo_{y}NPs in D2O at room temperature after purification.

FIG. 23 shows extinction at 360 nm vs. AuₓCo_{y}NP concentration for five independently synthesized batches of AuₓCo_{y}NPs (y=48.1±2.7%) with 5 dilutions for each sample set. The molar extinction coefficient was found from the slope of a linear regression of each data set according to Beer's Law. The molar extinction coefficient and associated error for each AuₓCo_{y}NP composition was found by taking the average and standard deviation, respectively, of all trials.

FIG. 24 shows emission spectra of AuₓCo_{y}NPs (y=48.1±2.7%) in water at 25.0° C. λ_{EX}=360 nm. The observed peak shoulder is due to solvent absorbance.

FIG. 25 shows maximum emission wavelength as a function of AuₓCo_{y}NP metallic core diameter. No correlation is observed (R²=−0.082).

FIG. 26 shows A) Plot of quantum yield (Φ) as a function of % Co incorporation as determined by ICP-MS. The linear regression was directly weighted with respect to the errors, R²=−0.093. B) Quantum yield as a function of AuₓCo_{y}NP metallic core diameter. No correlation is observed (R²=0.558).

FIG. 27 shows A) Plot of the molar extinction (E) as a function of % Co incorporation as determined by ICP-MS. The linear regression was directly weighted with respect to the errors, R²=−0.198. B) Molar extinction as a function of AuₓCo_{y}NP metallic core diameter. No correlation is observed (R₂=−0.172).

FIG. 28 shows A) Plot of the brightness (ε×Φ) as a function of % Co incorporation as determined by ICP-MS. The linear regression was directly weighted with respect to the errors, R²=−0.125. B) Brightness as a function of AuₓCo_{y}NP metallic core diameter. No correlation is observed (R²=−0.056).

FIG. 29 shows normalized excitation spectra of AuₓCo_{y}NPs in water at 25.0° C. λ_{EM}=950±40 nm.

FIG. 30 shows linear regression plots for AuₓCo_{y}NPs (y=48.1±2.7%) relaxivity at 37° C. as a function of per-Co concentration at A) 7 T and B) 0.47 T, and per-particle concentration at C) 7 T and D) 0.47 T. All R² values for the linear regression are >0.99. X and y error bars represent the standard deviation of concentration from ICP-MS and relaxation rates, respectively for three independently synthesized samples of the same initial molar ratio of Co.

FIG. 31 shows A) a plot of Cu2p_{3/2} binding energy (eV) versus % Au incorporated (ICP-MS) for two-phase and one-phase; B) a plot of Au4f_{7/2} binding energy (eV) versus % Au incorporated (ICP-MS) for two-phase and one-phase.

FIG. 32 (A)-(F) show plots of normalized counts/s versus kinetic energy for various percentages of Cu in two-phase and one-phase.

### DETAILED DESCRIPTION

It is to be understood that the descriptions of the present disclosure have been simplified to illustrate elements that are relevant for a clear understanding of the present disclosure, while eliminating, for purposes of clarity, other elements that may be well known. Those of ordinary skill in the art will recognize that other elements are desirable and/or required in order to implement the present disclosure. However, because such elements are well known in the art, and because they do not facilitate a better understanding of the present disclosure, a discussion of such elements is not provided herein. Additionally, it is to be understood that the present disclosure is not limited to the embodiments described above, but encompasses any and all embodiments within the scope of the description and the following claims.

In a typical experiment, AuₓCo_{y}NP alloys were synthesized by co-reducing various molar ratios of HAuCl₄ and Co(NO₃)₂ at room temperature in an aqueous solution containing a biologically compatible capping ligand, poly(ethylene glycol) methyl ether thiol (PEGSH, average Mₙ=1000 Da) with NaBH₄. The initial molar ratio of Co was varied from 0-100%, while maintaining the same total metal, capping ligand, and reducing agent concentrations. Due to the large deviation of % Co incorporated in the final particle composition at low values of % Co added during synthesis (Table 1), the initial molar ratio was finely adjusted to allow for tunability across composition space. The AuₓCo_{y}NP alloys were characterized with UV-visible spectroscopy, inductively coupled plasma mass spectrometry (ICP-MS), X-ray photoelectron spectroscopy (XPS), PL, HRTEM techniques, and ¹H nuclear magnetic resonance (NMR) techniques. Relevant physical properties of AuₓCo_{y}NPs are listed in Table 1.

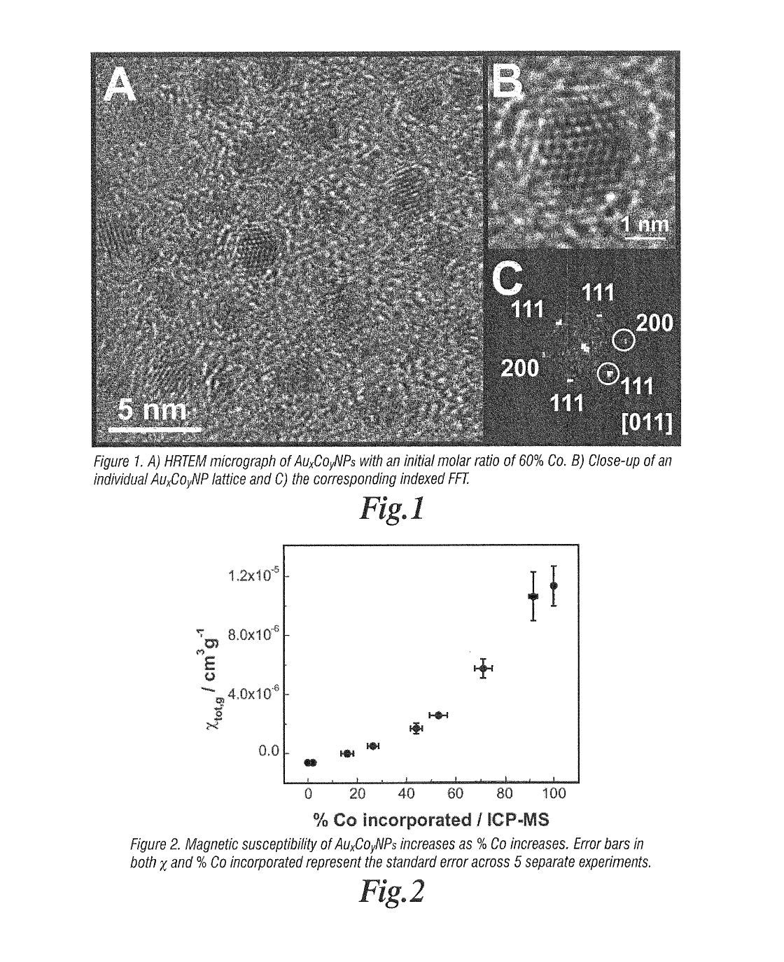

High-resolution transmission electron micrographs (HRTEMs) for Co₆₀Au₄₀PEGSH (initial molar ratios) and the corresponding fast-Fourier transform (FFT) are shown in FIG. 1. HRTEM analysis shows that all AuₓCo_{y}NPs are discrete, single crystalline nanoparticles (FIG. 1). All compositions of AuₓCo_{y}NPs were found to be pseudospherical in shape and the NP diameter was consistently found to be between 2.1-2.3 nm (with the exception of Co₁₀₀PEGSH) with a standard deviation of <20%.

TABLE 1
Physical characterization of AuₓCo_{y}NPs.

| | NP | Lattice | | NP size | ε at | | | | |
|---|---|---|---|---|---|---|---|---|---|
| Initial | composition | constant | NP size | (nm) | 360 nm | | | Magnetic | r₂ |
| mol | (% Co) | (Å) | (nm) | PFGSE- | (×10⁵ | Φ | Brightness | susceptibility | (mM_{NP}⁻¹s⁻¹) |
| (% Co) | ICP-MS | HRTEM | HRTEM | NMR | M⁻¹cm⁻¹) | (×10³) | (M⁻¹cm⁻¹) | (×10⁻⁷ cm³/g) | 0.47 T/7 T |
| 0 | 0 ± 0 | 3.96 ± 0.05 | 2.2 ± 0.5 | 5.0 ± 1.9 | 7.7 | 3.9 | 2997 | −6.5 ± 0 | NA |
| 40 | 2.8 ± 0.4 | 3.85 ± 0.03 | 2.3 ± 0.5 | 5.4 ± 1.6 | 9.1 | 4.3 | 3912 | −6.3 ± 0.2 | NA |
| 50 | 12.8 ± 1.1 | 3.70 ± 0.03 | 2.2 ± 0.2 | 4.7 ± 0.2 | 27.7 | 2.5 | 7041 | −0.4 ± 2.1 | 12/49 |
| 60 | 25.9 ± 2.4 | 3.75 ± 0.04 | 2.3 ± 0.5 | 4.1 ± 0.1 | 11.7 | 2.8 | 3278 | 4.6 ± 1.1 | 14/209 |
| 80 | 51.9 ± 3.8 | 3.73 ± 0.03 | 2.2 ± 0.3 | 4.7 ± 0.4 | 8.6 | 2.3 | 1961 | 16.3 ± 3.6 | 21/1750 |
| 85 | 58.6 ± 4.1 | | 2.1 ± 0.2 | 4.5 ± 1.6 | | 1.6 | | 25.1 ± 0.8 | NA |
| 90 | 71.6 ± 3.3 | 3.65 ± 0.06 | 2.2 ± 0.4 | 4.3 ± 0.1 | 5.6 | | | 56.6 ± 6.5 | 47/3650 |
| 95 | 91.2 ± 1.1 | 3.90 ± 0.06 | 2.3 ± 0.5 | 4.3 ± 0.1 | 3.3 | NA | NA | 105.5 ± 16.5 | NA |
| 100 | 100 ± 0 | 4.79 ± 0.05 | 2.9 ± 0.5 | 4.9 ± 0.1 | 2.6 | NA | NA | 112.6 ± 13.4 | 109/12200 |

*All reported values are the average of at least3 independently synthesized trials. The error bars for NP size represent he standard deviation of the mean. All other error bars represent the standard error.

Nanoparticle size and shape has been shown to have a dramatic influence on optical and magnetic properties. For example, PL of pseudospherical gold nanoparticles is observed in the NIR region at when the diameter is between 2-3 nm (λₑₘ ˜950 nm), but emits in the visible range (λₑₘ ˜700 nm) when the diameter is increased to ˜5 nm. Additionally, per particle T₂ relaxivity values have been shown to increase as particle diameter and surface faceting increases. Therefore, care was taken to thoroughly characterize nanoparticle size using several techniques. HRTEM was used to evaluate the shape and size distribution of the NP metallic core, and ¹H pulsed field gradient stimulated echo (PFGSE) NMR was used to measure the hydrodynamic diameter of the nanoparticles. All compositions of AuₓCo_{y}NPs were found to have similar sizes for both measurements (Table 1). From the ¹H PFGSE NMR measurements, the thickness of the passivating PEGSH layer was found to be between 0.9-1.5 nm for all NP compositions, consistent with Mₙ=1000 Da.

The average incorporation percentage of Au and Co into the final particle for the entire colloid of each AuₓCo_{y}NP composition was analyzed by ICP-MS (Table 1) and XPS. Little to no cobalt incorporation was observed via ICP-MS in the final NP until the initial molar ratio of Co was increased to 40%. We believe that this initial lag in Co incorporation is the result of the large disparity in reduction potential for Co (E⁰=−0.28 V) compared to Au (E⁰=+1.50 V), resulting in less available Co⁰ after reduction with NaBH₄. Therefore, to achieve tunability in the final NP composition, the initial molar ratio of Co was adjusted accordingly. Previous reports indicate that co-reduction is extremely important to allow nucleation of both metal monomers in a single particle. Even in the event of rapid reduction, differences in reduction potential have been shown to result in the formation of core-shell particles or incomplete mixing of the two components. After 40% Co initial molar ratio, a small amount of Co⁰ monomer co-nucleates along with Au⁰, allowing both elements to be incorporated into one particle. Investigation into the mechanism of alloy formation is ongoing in our laboratory.

Additional composition information of individual AuₓCo_{y}NPs was characterized by scanning transmission electron microscopy-energy dispersive X-ray spectroscopy (STEM-EDS). To confirm that both Co and Au were present in a single particle (i.e. rule out that individual monometallic particles of pure Co and pure Au were not produced in the synthesis), STEM-EDS analysis was performed on select particle compositions (SI). For the AuₓCo_{y}NP compositions analyzed, both elements were present in a single particle. Also, HRTEM analysis shows evidence of single crystalline particles, indicating the formation of solid-solution NP alloys. No evidence of core-shell or Janus structures are observed.

Further, HRTEM analysis of the lattice fringes of each AuₓCo_{y}NP composition shows evidence for alloying (FIG. 1). Considering that the bulk lattice constant of Au=4.08 Å and the bulk lattice constant of metallic Co=2.51 Å for hcp and 3.50 Å for fcc, it is expected that as the % Co increases, the lattice constant will decrease. Although the lattice constants for even pure Au particles (Table 1) are smaller than the bulk value, due to lattice strain as a result of the high radius of curvature, there is a general decrease in lattice constant as the % Co increases. However, once the % Co incorporation reaches a threshold (>90%) the lattice constant begins to increase. For 100% CoNPs, the lattice constant is similar to that of cobalt oxide (CoO=4.26 Å, Co₂O₃ a=4.64 c=5.75 Å, and Co₃O₄=8.05 Å), indicating the presence of cobalt oxidation in mixture containing >90% Co (e.g. Co₉₅Au₅PEGSH and Co₁₀₀PEGSH). This characterization scheme confirmed that we synthesized small (d=2-3 nm), discrete, AuₓCo_{y}NP alloys with tunable composition.

FIG. 1(A) shows an HRTEM micrograph of AuₓCo_{y}NPs with an initial molar ratio of 60% Co. FIG. 1(B) is a close-up of an individual AuₓCo_{y}NP lattice and FIG. 1(C) the corresponding indexed FFT.

To determine the influence of % Co incorporation on the magnetism of AuₓCo_{y}NPs, we used the Evans' NMR method to measure the mass magnetic susceptibility. The Evans' NMR method is an attractive method to rapidly evaluate magnetic properties, of nanoparticles in particular, because it is performed in solution (the entire colloid is analyzed), does not require a high concentration of material, is relatively simple, and widely accessible. With this method, we found that by altering the % Co incorporated in the final AuₓCo_{y}NP we could achieve tunable susceptibility (FIG. 2). The reported magnetic susceptibility values (Table 1) represent the total magnetic susceptibility of the sample, which is comprised of the diamagnetic contribution and the paramagnetic contribution. By using a molecular characterization method to analyze our magnetic susceptibility, we were able to directly observe the ¹H NMR spectrum of our ligand shell in each sample with one experiment. This allowed us to efficiently assess and reduce variability in magnetic susceptibility measurements. For example, we confirmed that contaminant inferences were minimized by the purification procedure, which ensured that residual reactants, including metal salts, free capping ligand, and reducing agent were removed. ¹H NMR spectra show no evidence of free PEGSH (upon attachment to the NP surface, the thiol proton as well as the CH₂ protons adjacent to the thiol are absent from the spectra due to significant dephasing as a result of chemical shift distribution and conduction electrons at the particle surface). Control experiments were performed to ensure that changes in magnetic susceptibility and relaxivity were not the result of free Co²⁺ cations or other excess reactant impurities. The ability to observe the NMR spectra of the ligand shell, while assessing the physical properties of the colloid, provides the platform to quantitatively analyze the magnetic (Evans' method), dynamic (i.e. relaxation experiments as well as dipolar coupling measurements), and structural (i.e. 2D correlation experiments) properties of materials.

FIG. 2 shows magnetic susceptibility of AuₓCo_{y}NPs increases as % Co increases. Error bars in both χ and % Co incorporated represent the standard error across 5 separate experiments.

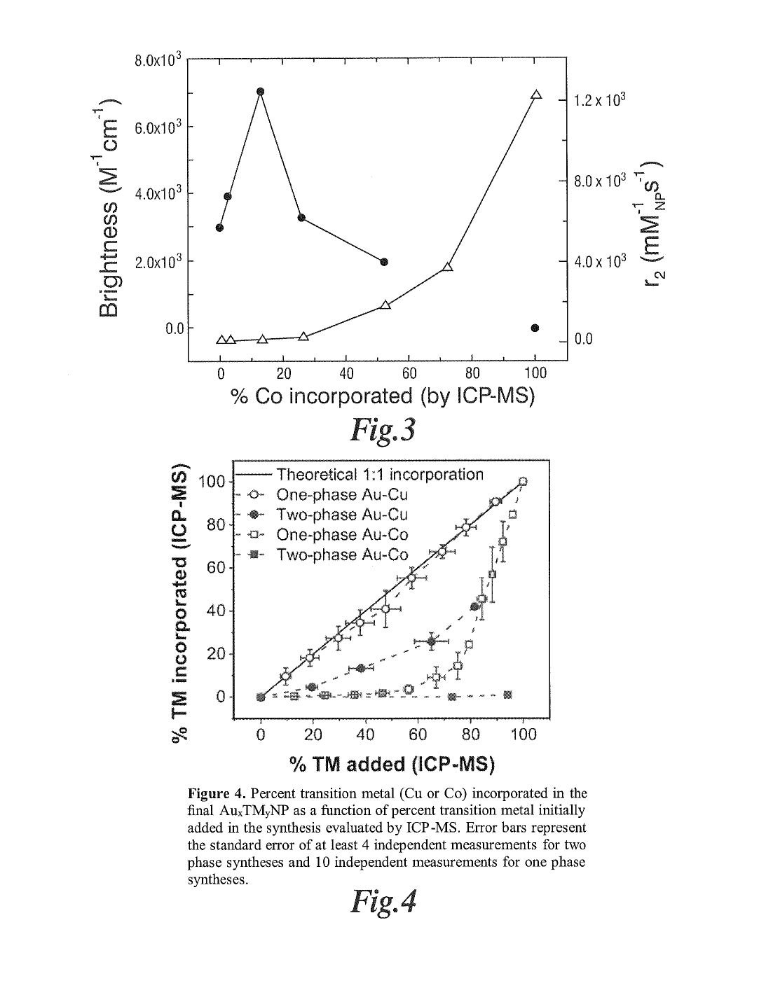

Once the magnetic properties of the AuₓCo_{y}NPs had been fully characterized, we examined the photoluminescent behavior. The alloying of transition metals (TM) with Au in this size regime (d=2-3 nm) has been found to have an impact on the optical properties of AuₓTM_{y}NPs. In particular, the emission maxima, quantum yield, and brightness were found to strongly dependent on NP composition independent of NP size. For this reason, we carefully measured the PL properties of the AuₓCo_{y}NPs in D₂O to minimize solvent interference. All of the compositions listed in Table 1 exhibit NIR PL, with the exception of Co₁₀₀PEGSH. We note that although Co₉₅Au₅PEGSH showed weak PL in the NIR, the poor signal-to-noise precludes its use as an imaging tool. As shown in the spectra of AuₓCo_{y}NPs in FIG. 3, although all compositions exhibit NIR PL, the emission maxima does not change as a function of composition. Quantum yield (Φ) and molar extinction coefficient (ε) measurements (details in SI) were used to calculate particle brightness (brightness=ε×Φ). Brightness measurements are used to determine the probability of absorption and emission of photons and is an especially useful figure to evaluate new materials. Even though composition did not alter the maximum emission wavelength, we found that the quantum yield and brightness varied non-linearly (values listed in Table 1) as a function of composition, consistent with previous reports.

The quantum yield values reported are consistent with those found for other noble metal nanoparticle systems. Similar to AuₓCu_{y}NP alloys at this diameter, the AuₓCo_{y}NP alloys also exhibit exceptional brightness. When evaluated in D₂O, the Co₅₀Au₅₀NPs display brightness values that are nearly two orders of magnitude higher (7041 M⁻¹cm⁻¹ vs. 83 M⁻¹cm⁻¹) than the brightest sensitized lanthanide complex, (Yb(III)TsoxMe)). Across compositions, the AuₓCo_{y}NP brightness is ideal for NIR imaging purposes.

The observation of tunable magnetic susceptibility and NIR brightness across a range of AuₓCo_{y}NPs provided evidence that these particles could have an application as MRI contrast agents, equipped with bimodal imaging capabilities. Previous reports indicate that Co T₂ relaxivities are field-strength, as well as, concentration dependent. To study the effect of field strength, the relaxivity of the AuₓCo_{y}NPs was measured at 37° C. at two different static fields, 0.47 T (20 MHz proton Larmor frequency) and 7 T (300 MHz proton Larmor frequency). For both field strengths, we found that the AuₓCo_{y}NPs greatly affect the transverse relaxation time (T₂) and have little to no effect on the longitudinal relaxation time (T₁). This means that AuₓCo_{y}NPs have the ability to maintain proton T₁ values the same as the surrounding tissue (providing essentially no positive contrast) while significantly dephasing the transverse magnetization utilized in MRI signal detection. This property most efficiently produces negative (dark) spots in the final image, making the AuₓCo_{y}NPs reported here are applicable as a negative-T₂ contrast agents.

In fact, even at low field strength, all AuₓCo_{y}NP compositions show very little effect on T₁, leading to r₂/r₁ values that, in all cases, are either comparable to or larger than those of a clinically available T₂ contrast agent, Ferumoxsil (which has a diameter nearly 3 times larger). The comparable, or in some cases, enhanced relaxivity for AuₓCo_{y}NPs, despite their smaller diameter compared to reported iron oxide NPs is likely the result of the higher saturation magnetization of Co compared to iron oxide. However, AuₓCo_{y}NP magnetization is field strength dependent, resulting in r₂ values at 0.47 T that are rather low. Since tissues already have relatively short T₂ times (˜10²-10³ ms), to be considered a safe, effective negative T₂ contrast agent, r₂ values must be orders of magnitude larger than r₁ values typically required for positive contrast agents. As clinical instruments move to higher field strengths to increase resolution, the values found at 0.47 T represent the lower bound of relaxivity exhibited by these particles. Research laboratory, and even some clinical settings, routinely perform imaging at 7 T or greater.

As field strength is increased, T₁ effects, as well as the efficiency of positive contrast agents, are expected to diminish. As predicted, at 7 T the longitudinal relaxation time for even the most concentrated AuₓCo_{y}NPs with the highest susceptibility is equal to that of water (˜6 s at 7 T). For this reason, only the T₂ relaxivities at 7 T (r₂ values at 7 T are listed in Table 1) are used to evaluate the optimal composition for bimodal imaging. Relaxivity values are reported in Table 1 as per-particle relaxivities to facilitate accurate comparison between nanoparticles of different composition and size. The efficiency of MRI contrast agents is evaluated by the event of water interaction with the contrast material. For chelated-metal contrast agents, such as commercially available gadolinium based agents, water protons bind to a single metal center, and therefore, the “per-particle” number of metals is one and per-metal relaxivities are commonly reported. For superparamagnetic nanoparticles, the particle itself behaves as a large paramagnetic ion. Therefore, per-particle relaxivities provide an accurate assessment of contrast agent efficiency in the case of nanoparticles.

To compare AuₓCo_{y}NP T₂ relaxivities to other contrast agents, the per-particle relaxivity was calculated for reported abundant-earth metal nanoparticles of comparable size. We found that per-particle relaxivities for 100% CoNPs (d_{core}=2.9 nm, d_{H}=4.9 nm, r₂=12200 mM_{NP}⁻¹s⁻¹) were comparable to the larger (d_{core}=8.4 nm, d_{H}=300 nm), pharmaceutically available Ferumoxsil (r₂=13000 mM_{NP}⁻¹s⁻¹). The 100% CoNPs exhibit T₂ relaxivities the same order of magnitude, though slightly smaller when compared to more recent PEG-capped Fe₃O₄ particles of comparable size (d_{core}=3 nm, r₂=27890 mM_{NP}⁻¹s⁻¹), indicating the important role of surface chemistry. Both Co₉₀Au₁₀PEGSH and Co₈₀Au₂₀PEGSH possessed relaxivities of the same order of magnitude as larger iron oxide particles, marketed as Ferumoxtran (d_{core}=4.9 nm, d_{H}=49.7 nm, r₂=2590 mM_{NP}⁻¹s⁻¹) and Ferumoxide (d_{core}=4.8 nm, d_{H}=227 nm, r₂=2430 mM_{NP}⁻¹s⁻¹). When compared to PEG-1000-coated iron oxide particles of similar diameter (d_{core}=1.9 nm, r₂=150 mM_{NP}⁻¹s⁻¹), the per-particle relaxivities for Co₆₀Au₄₀PEGSH are slightly larger (d_{core}=2.3 nm, r₂=209 mM_{NP}⁻¹s⁻¹), while higher % Co compositions are larger by over an order of magnitude. These properties make AuₓCo_{y}NPs suitable as negative contrast agents. When compared to novel 0D and 1D DNA-templated Au—CoFe₂O₄ nanostructures, AuₓCo_{y}NPs exhibit T₂ relaxivity values that are over an order of magnitude larger when y>60%.

In order to determine the optimal composition at which the AuₓCo_{y}NPs exhibit both high brightness and high relaxivity, we plotted r₂ at 7 T and NIR brightness as a function of % Co incorporation (FIG. 3). From this plot we can see that particle brightness reaches the highest value Co₅₀Au₅₀PEGSH (initial molar ratio) and steadily drops until no NIR PL is observed. For per-particle relaxivity, as % Co incorporated increases, r₂ values become more favorable for negative MRI contrast. However, we have established that the per-particle relaxivity for Co₈₀Au₂₀PEGSH remains competitive when compared to other negative contrast agents and exceeds the relaxivity values for iron oxide nanoparticles of similar sizes. At this composition, the particle brightness (1961 M⁻¹ cm⁻¹) also remains relatively high when compared to other NIR probes. For this reason, we conclude that Co₈₀Au₂₀PEGSH (initial molar % Co=80%, actual % Co incorporated=52%) is the optimal composition for a dual NIR-T₂ weighted imaging agent.

FIG. 3 shows an example of the optimal composition for bimodal NIR-T₂ weighted MRI imaging occurs at Co₈₀Au₂₀PEGSH (actual % Co incorporation ˜50%).

In summary, we have described the synthetic framework to optimize bimodal properties in small diameter (d=2-3 nm), discrete, composition-tunable nanoparticle alloys. The AuₓCo_{y}NP alloys reported here offer exemplary physical properties that allow the improvement and expansion of imaging techniques and medical diagnostics with bimodal capabilities. These materials provide the opportunity to not only access new bimetallic combinations, inaccessible on the bulk scale, but the ability to rationally optimize multimodalities within a single particle.

The AuₓCo_{y}NPs are the first report of tunable gold-cobalt alloys across all composition space, overcoming bulk immiscibility limitations.

Both Co₉₀Au₁₀PEGSH and Co₈₀Au₂₀PEGSH exhibit relaxivities of the same order of magnitude as larger iron oxide particles, marketed as Ferumoxtran (d_{core}=4.9 nm, d_{H}=49.7 nm, r₂=2590 mMNP−1s−1) and Ferumoxide (d_{core}=4.8 nm, d_{H}=227 nm, r2=2430 mM_{NP}⁻¹ₛ₋₁).

When compared to PEG-1000-coated iron oxide particles of similar diameter (d_{core}=1.9 nm, r₂=150 mM_{NP}⁻¹s⁻¹), the per-particle relaxivities for Co₆₀Au₄₀PEGSH are slightly larger (d_{core}=2.3 nm, r₂=209 mM_{NP}⁻¹s⁻¹1), while higher % Co compositions are larger by over an order of magnitude.

When compared to novel 0D and 1D DNA-templated Au—CoFe2O4 nanostructures, AuₓCo_{y}NPs exhibit T₂ relaxivity values that are over an order of magnitude larger when y>60%.

When evaluated in D₂O, the Co₅₀Au₅₀NPs display brightness values that are nearly two orders of magnitude higher (7041 M⁻¹cm⁻¹ vs. 83 M⁻¹ cm⁻¹) than the brightest sensitized lanthanide complex, (Yb(III)TsoxMe)). Across compositions, the AuₓCo_{y}NP brightness is ideal for NIR imaging purposes.

Overall, by altering the nanoalloy composition, we found the optimal composition, Co₈₀Au₂₀NP (initial molar ratio Co:Au=80:20) that exhibits both exceptional brightness and high per-particle relaxivity for use as a dual NIR-T₂-weighted contrast imaging agent.

Known alternative methods and related developments by others in this field include: marketed iron oxide nanoparticles; recently developed iron oxide nanoparticles of a similar size regime; 0D and 1D DNA-templated Au—CoFe₂O₄ and Au—Fe₂O₃ nanostructures; composition-tunable gold-cobalt nanoparticle alloys with tunable NIR emission; and noble metal nanoparticle systems that exhibit photoluminescence properties.

### Manipulating Precursor Chemistry to Control Atom Position and Incorporation Behavior in Small Gold-Transition Metal Nanoparticle Alloys.

Several studies on bimetallic noble metal nanoparticles have demonstrated that in addition to composition, relative atom position of the constituent elements is crucial to manipulating the observed electronic structure. For example, recent reports have demonstrated that the relative position of Cu in Au—Cu nanoparticles at both the small (25 atoms) and large (144-145 atoms) end of the cluster regime can have a dramatic effect on the optoelectronic properties. Here, absorbance spectra can serve as a readout for the perturbation the heteroatom induces on the electronic structure of the underlying particle. Therefore, ability to control atom position in bimetallic nanostructures is highly desirable because it provides the opportunity to tune the electronic structure of the particle for applications ranging from energy storage to heterogeneous catalysis.

In order to achieve the vision of atomistic control of bimetallic nanostructures, we must provide an understanding of metal mixing behavior at the nanoscale by employing methods that provide molecular resolution of the precursors at the beginning stages of metal mixing. Recent work describing the molecular evolution of precursor materials for monometallic noble metal nanoparticles and quantum dot materials shows deviation from the expected behavior of classic nucleation theory with the observation of stable “pre-nucleation” species. The existence of pre-nucleation species is intriguing because it suggests the possibility of multiple-step nucleation or aggregation-induced particle formation pathways, and may also provide a route to understand nanoscale metal mixing behavior and deviations from bulk metal mixing trends.

Here, we characterize and compare the different precursor species present in solution prior to reduction in two common synthetic routes to achieve small (diameter, d=1-3 nm), mono- and bimetallic thiol-capped nanoparticles. Precursor characterization via high resolution NMR spectroscopic techniques, XPS, X-ray absorption spectroscopy (XAS), and matrix assisted laser desorption ionization time-of-flight mass spectrometry (MALDI-TOF-MS) revealed one of the first observations of bimetallic nanoparticle precursors in thiolated nanoparticle synthesis. With distinguishable nanoparticle precursors in hand, we systematically evaluate the impact of precursor chemistry on final bimetallic nanoparticle architecture and nanoparticle formation. The final nanoparticles are characterized with XPS, ICP-MS, high resolution transmission electron microscopy (HRTEM), scanning transmission electron microscopy-energy dispersive X-ray spectroscopy (STEM-EDS), XAS, and ¹H NMR spectroscopy. We investigate two binary metal systems, Au—Cu and Au—Co, in order to probe metal mixtures that do and do not exhibit bulk miscibility at room temperature, respectively.

The influence of precursor chemistry on final bimetallic nanoparticle architecture was investigated by comparing two ambient preparation methods for small (d=1-3 nm), thiolated metal nanoparticles. The first method is a one-phase aqueous synthesis in which a 1:1 molar ratio of metal salt:thiolated ligand are reduced with sodium borohydride. In the second approach, we use a standard Brust-Schiffrin synthesis where aqueous metal salts are transferred into toluene via a phase transfer agent, tetraoctylammonium bromide. From here, a thiolated ligand is introduced and the reaction mixture is reduced with aqueous sodium borohydride.

Initial examination of the gold-transition metal nanoparticle (AuₓTM_{y}NP) compositions as a function of initial molar ratio revealed interesting trends, both as a result of preparation method and choice of transition metal (FIG. 4). In the case of Au—Cu, the one-phase approach exhibits incorporation behavior that reflects the molar ratio that is fed into the synthesis. However, if Au—Cu is synthesized using the two-phase approach, deviation from 1:1 incorporation behavior is observed, with less Cu incorporated in the final particle than is initially present. The difference in Cu incorporation into the AuₓCu_{y}NP between the one- and two-phase syntheses indicated that a possible difference in particle formation pathway exists.

Likewise, the one-phase synthesis of AuₓCo_{y}NPs shows no incorporation in the final nanoparticle under 50% Co. After this point, continuous composition tunability is observed. Curiously, all attempts to prepare Au—Co metal mixtures via the two-phase approach were unsuccessful, despite high initial molar ratios of Co in the initial synthesis, further suggesting mechanistic differences between the two methods. Taken together, the AuₓTM_{y}NP composition evaluated by ICP-MS indicate that drastic differences in nanoparticle formation exist between the one-phase and two-phase methods, as well as the choice of transition metal.

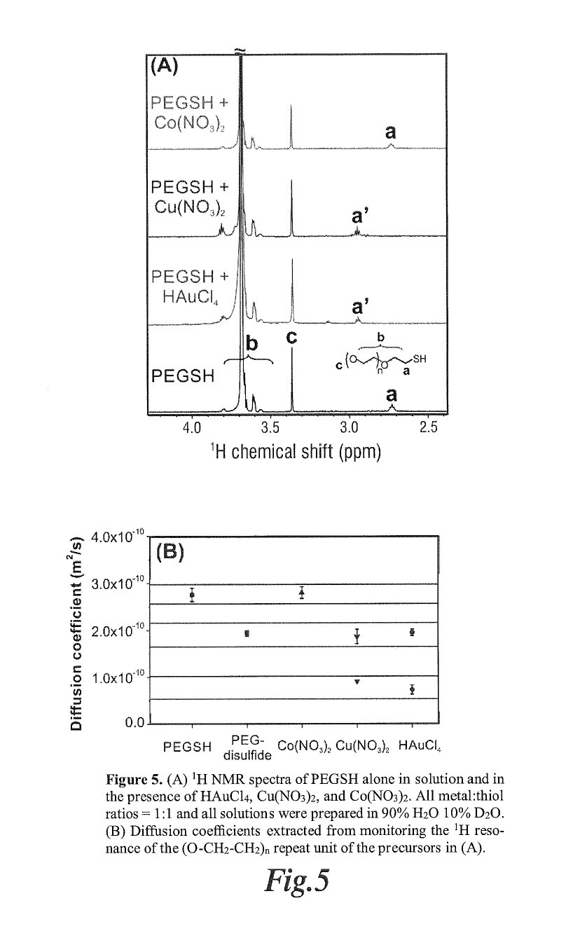

We hypothesized that the differences observed between the AuₓTM_{y}NP results shown in FIG. 4 were the result of differences in precursor chemistry that ultimately dictates differences in particle formation pathways and final bimetallic nanoparticle architecture. To determine the molecular differences in precursor between the two methods, 1D ¹H NMR and pulsed field gradient techniques were used to provide a atomistic detail of the precursor species present in solution prior to reduction with sodium borohydride at synthetic concentrations (FIGS. 5A and 5B). Further characterization using XPS, MS, and density functional theory (DFT) was also performed.

One-Phase Precursors.

In one-phase syntheses, the water-soluble, thiolated ligand used was poly(ethylene) glycol methyl ether thiol (PEGSH, =1000). In the presence of only Co(NO₃)₂, the chemical shifts and corresponding integrals of PEGSH ¹H resonances are similar to PEGSH alone in solution. Given that Co(II) is paramagnetic, any binding to the PEGSH would result in dephasing of nearby resonances and/or large changes in chemical shift, depending on the Co(II) spin state. The absence of any line broadening along with no changes in chemical shift and integration values indicates that Co(NO₃)₂ does not interact with the PEGSH at a timescale observable via NMR.

In contrast, reaction mixtures containing HAuCl₄ and Cu(NO₃)₂ show oxidation products of the thiol-group of the PEGSH molecule. Oxidation of the thiol functionality is evidenced by the shift of the triplet from the protons adjacent to the sulfur from 2.6 ppm (¹H adjacent to a thiol group) to 2.9 ppm (¹H adjacent to a disulfide). Mixtures containing PEGSH and Cu(NO₃)₂ show that the furthest oxidation product is the disulfide, whereas in the presence of HAuCl₄, an additional triplet peak at 3.1 ppm arises, suggesting further oxidation products.

To further probe the origin of this peak, ligand oxidation products as a function of Au:thiol were measured with ¹H NMR. Even at low % Au, oxidation of the thiol functionality on the PEGSH is evidenced from the appearance of the triplet at 2.9 ppm. As the concentration of HAuCl₄ is increased, the amount and degree of thiol oxidation products also increases. In reaction mixtures containing >50% Au, further oxidation of the PEG-disulfide to a sulfonic acid derivative can observed as the triplet that appears at 3.1 ppm. ¹H-¹³C HSQC analysis provided additional evidence of the oxidation of the thiol group to a disulfide, and eventually conversion to a sulfonic acid, in the presence of HAuCl₄. Further oxidation of the disulfide moiety to a sulfenic-, sulfonic-, and sulfonic-acid derivative is consistent with previous reports on HAuCl₄ oxidation of disulfide groups. Importantly, at molar ratios of Au:thiol, in which the Au(III) oxidizes the disulfide to the sulfonic acid derivative, Au⁰ is formed as the corresponding reduction product, as inferred from both ligand oxidation products and X-ray analysis (vide infra).

Curiously, the sulfonic acid peak appears at >50% Au in the reaction mixture is the same molar ratio where little to no Co incorporation is observed (area of lag time for % Co incorporation in FIG. 4). This observation suggests that % Co incorporation could be inhibited because a cross over from homogeneous to heterogeneous nucleation may occur at >50% Au at the metal:thiol ratios in our reaction conditions.

In the case of Cu(NO₃)₂ and HAuCl₄, ¹H diffusion measurements indicated that high molecular weight species were present in solution (FIG. 5), consistent with the formation of a metal-thiolate structure as a result of the redox process. On the other hand, Co(NO₃)₂, which exhibited no redox chemistry with the thiol, showed no change in PEGSH diffusion coefficient. Calibration with molecular weight standards indicates that the mass of the slow diffusing component is approximately ˜4-5 kDa.

Once the ligand chemistry was assigned by NMR, the respective metal precursor oxidation states prior to reduction with NaBH₄ were analyzed in both the solid state and solution phase with XPS and XAS, respectively. XPS analysis was consistent with NMR spectroscopic evidence that a redox process occurred between PEGSH and Cu(NO₃)₂ and HAuCl₄ before addition of an external reducing agent. XPS analysis of the Au4f region for all precursors indicate that upon exposure to PEGSH, all of the HAuCl₄ is reduced primarily to a Au(I) as evidenced by the chemical shift at ˜84.5 eV, consistent with the XAS findings in the solution phase.

Here, EXAFS analysis showed that all of the HAuCl₄ is present primarily in a Au(I)—S bonding motif. Careful analysis of the Au L3-edge indicates that the mixed Au—Cu precursor has similar near-edge structure to that of Au(I)-alkanethiolates. The monometallic HAuCl₄+PEGSH precursor exhibited similar Au L3 near-edge structure as the bimetallic Au—Cu precursor solution, with the exception of decreased whiteline intensity, indicating the presence of a small amount of metallic Au(0). However, no long range order was observed in the Au EXAFS analysis. This observation is consistent with the NMR observation of higher order sulfur oxidation products that would produce a small amount of Au(0) (the redox reaction between Au(III) and disulfides is known to produce sulfonic acid derivatives and metallic Au). We hypothesize that no long range order is observed in the EXAFS because the Au(0) is just beginning to form critical nuclei, possibly even composed of only a few atoms.

Curiously, examination of the Cu2p XPS region showed that in both mixed Au—Cu and 100% Cu metal nanoparticle precursor solids, only a fraction of the Cu(NO₃)₂ is reduced from the initial Cu(II) to the Cu(I) oxidation state. Quantification based on peak fitting, integration, and empirical sensitivity factors associated with Cu(I) species indicated that only ˜12% of Cu(II) was reduced to Cu(I) in the case of the monometallic 100% Cu(NO₃)₂ precursors. Likewise, in the case of the 50:50 Au:Cu bimetallic precursors only ˜17% of Cu(II) was reduced to Cu(I), resulting in an overall approximate initial molar ratio of 50% Au(I), 9% Cu(I), and 41% Cu(II). Similarly, XAS analysis of the Cu scattering paths are also consistent with only a small amount (˜5-10%) of Cu(I) present and participating in a Cu(I)—S bond. Unfortunately, due to the lack of long range order in these structures, further information on the architecture of the metal-thiolate precursors was difficult to ascertain from XAS analysis alone. Therefore, to provide further insight into the structure and morphology precursors, we performed MALDI-TOF-MS analysis of the pre-nucleation species along with density function theory (DFT) calculations.

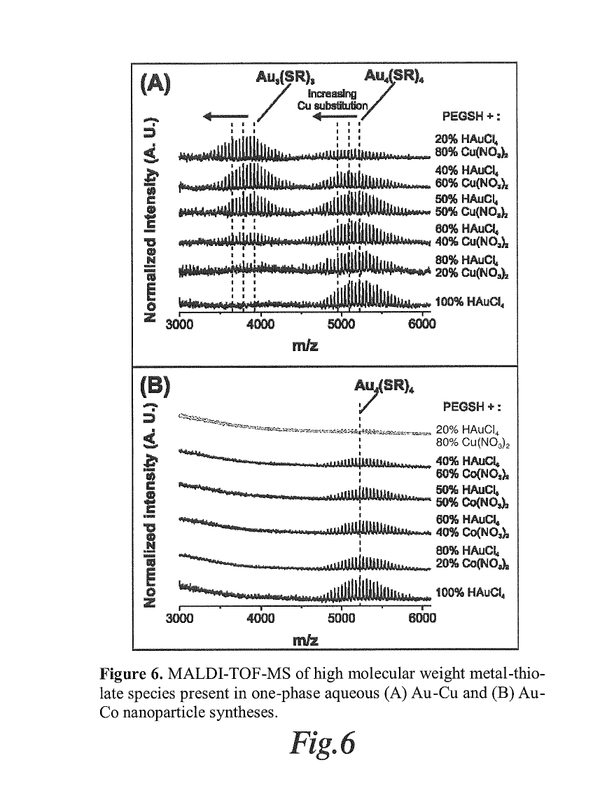

To provide a more robust structural assignment of the composition and size of the pre-nucleation species, a range of Au:Cu initial molar ratio pre-nucleation species were prepared and analyzed with MALDI-TOF-MS. MALDI-TOF-MS was used to probe both the size of the pre-nucleation species and the composition (FIG. 6). In the case of 100% HAuCl₄+PEGSH, a high molecular weight species is observed with a center of mass at 5222.5 m/z. It is important to note that consistent with NMR analyses, large populations of lower molecular weight species consistent with PEG-disulfide (and higher oxidation products in the case of HAuCl₄) are also observed but are not shown for clarity. In all mass spectra, a broad distribution is observed that is consistent with the breadth expected from a species consisting of a combination of ligands with a molecular weight distribution of the original ligand, PEGSH (See SI for calculation of distribution breadth). Each peak is separated by 44 m/z units, which corresponds to the mass of one ethylene glycol unit (O—CH₂—CH₂), indicating that the species is in the +1 charge state. Comparison of MALDI-TOF-MS and ESI-MS of PEGSH indicates that PEGSH is present as the Na⁺ adduct in the MALDI-TOF-MS with a center of mass at 1129 m/z, whereas the mass of the ligand alone is 1104 Da.

Given this analysis, the high molecular weight distribution centered at 5222.5 m/z is consistent with the Na⁺ adduct of a Au₄(SPEG)₄ species, which would have a m/z=5222.9. As the initial molar ratio of Cu(NO₃)₂ is increased relative to HAuCl₄, a dramatic change in the MALDI-TOF-MS is observed (FIG. 6A). Even at a modest initial molar ratio of 20% Cu(NO₃)₂:80% HAuCl₄, an additional species emerges in the tetrameric species that appears to correspond to Au₄(SPEG)₄ and Au₃Cu₁(SPEG)₄. Unfortunately, the mass difference between Au and Cu (Δm=196.97-63.55=133.42) results in a mass that is close to a multiple of 44. Therefore, resolution between different Cu-substituted species is beyond the resolution capabilities of our MALDI-TOF mass spectrometer. Attempts were made to fit the individual species to Gaussian distributions but given the caveats with fitting several different unknown populations of mixed metal species to a sum of Gaussians as well as MALDI-TOF-MS data of mixtures of polymers with molecular weight distributions, the interpretation was largely subjective and is not used for quantitation compositional analysis.

As the initial molar ratio of Cu(NO₃)₂ is increased to >40%, an additional peak is observed in the MALDI-TOF-MS that corresponds to a trimeric species. At an initial molar ratio of 40% Cu(NO₃)₂:60% HAuCl₄, the molecular weight distribution of the trimeric species is centered at 3922.6 m/z, which corresponds to the Na⁺ adduct of a Au₃(SPEG)₃ species. Similar to the tetramer, as the initial molar ratio Cu(NO₃)₂ is increased, the center of mass of the molecular weight distribution of the trimeric species shifts to lower m/z and appears to correspond to a monometallic Au₃(SPEG)₃ and a bimetallic Au₂Cu₁(SPEG)₃ species with a center of mass at 3790.4 m/z. In general, as the initial molar ratio of Cu(NO₃)₂ is increased relative to HAuCl₄, the population of trimeric species increases and the % Cu substitution into the pre-nucleation species also increases. As expected, the only species observed for all Au:Co precursors is the Au₄(SPEG)₄ tetramer (FIG. 6B), consistent with other experimental evidence that no Co—S bonds are formed prior to NaBH₄ addition.

In order to provide further insight into the architecture and metal mixing behavior of the Au—Cu pre-nucleation species observed in NMR and MS, DFT calculations were performed. These simulations indicate that a ring-like structure is more stable than a linear architecture. In addition, notable differences in the stabilization energy between the monometallic Au-thiolate and Cu-thiolate structures are observed that may explain the shift from a tetrameric species to an increased population of trimeric species as the initial molar ratio of Cu(NO₃)₂ is increased. Also shows that mixed metal architectures are more energetically stable than segregated structures.

Taken together, these observations provide the first piece of evidence that metal-sulfur bond formation as well as metal mixing can begin prior to NaBH₄ addition in one phase bimetallic nanoparticle syntheses. DFT calculations further indicate that these mixed-metal pre-nucleation species are relatively stable in solution and exhibit a preference for size and metal-mixing behavior based on composition.

It is noted that characterization of metal-thiolates is notoriously difficult due to their insolubility in a variety of solvents and inability to form crystals suitable for X-ray analysis, with a few exceptions. Despite these difficulties, some soluble Au(I)-thiolates have been prepared as anti-arthritic pharmaceuticals and thoroughly characterized with NMR spectroscopy and X-ray crystallography. In addition, previous reports demonstrated that both synthetically prepared, soluble Au(I)-thiolates as well as Au(I)-thiolates observed as intermediates in nanoparticle syntheses could serve as rational precursors to gold nanoparticles.

However, the data presented here suggests that metal-sulfur bond formation occurs prior to NaBH₄ addition for both mono- and bimetallic one phase nanoparticle syntheses. The metal-thiolates observed in the one-phase syntheses described here behave differently than expected in the sense that they are soluble in aqueous solution, allowing characterizing by solution phase NMR and ample time for characterization by XAS, XPS, and MALDI-TOF-MS. We believe that this is due to the PEGSH functionality imparting increased solubility (and possibly favorable steric hindrance of aurophilic interactions, known to facilitate polymerization in solution) on the trimeric and tetrameric pre-nucleation species. Indeed, if we attempt to synthesize pre-nucleation species in aqueous solution with other small molecule thiolated ligands, a white precipitate is rapidly observed, indicating the formation of an insoluble metal-thiolate. The increased solubility is similar to that observed for zwitterionic, small molecule ligands such as thiomalate that compose anti-arthritic drugs such as myocrysin. Unfortunately, while imparting solubility, the polymeric nature of PEGSH prohibits crystallization for single crystal X-ray analysis of the pre-nucleation species.

Two-Phase Precursors.

The existence of the one-phase pre-nucleation structures as characterized indicates that (i) metal-sulfur bond formation occurs before NaBH₄ addition and (ii) metal mixing begins before the addition of NaBH₄ in one phase alloy syntheses. In order to test this hypothesis, we compared the bimetallic nanoparticle products yielded from a one-phase aqueous synthesis to a standard two-phase Brust-Schiffrin synthesis, in which metal-thiolate precursors were demonstrated not to form prior to nucleation. Indeed, ¹H NMR analysis of the two phase precursors revealed that contrary to the observations in the one-phase synthesis, no metal-sulfur bonds were formed prior to NaBH₄ addition. Rather, the synthesis proceeded as described previously, via metal halide anion coordination to the [TOA]⁺ inverse micelle surface for monometallic noble metal nanoparticle synthesis. In the first step, aqueous metal salts are transferred to the organic phase (toluene) by tetraoctylammonium bromide (TOAB) and the aqueous layer is removed. Here, the ¹H NMR spectra are consistent with metal anions (in this case, [AuX₄]⁻, [CuX₄]²⁻, and/or [CoX₄]²⁻, where X=Cl⁻ or Br⁻) binding to the quaternary ammonium headgroup of the [TOA]⁺, based on the chemical shift change of the nearby resonances, consistent with fast anion exchange between the metal halide complexes and the free halides. Further, in the case of the paramagnetic metal precursors, Cu(II) and Co(II), distance dependent ¹H signal dephasing is observed for resonances closest to the quaternary ammonium. This distance-dependent dephasing is apparent from the broadening of the resonances closest to the quaternary ammonium, whereas the terminal methyl remains narrow, consistent with the formation of a micellular structure.

Upon addition of dodecanethiol (DDT), the signal dephasing is eliminated in reaction mixtures containing Cu, indicating that the Cu(II) has been reduced to diamagnetic Cu(I). Further, the increased spectral resolution allows for the determination of anion composition on the micelle surface. Comparison between reaction mixtures containing 100% Au, 50:50 Au:Cu, and 100% Cu shows a gradual shift in the ¹H resonance adjacent to the quaternary ammonium, suggesting a change in anion composition on the micelle surface. Assuming that the halide concentration does not change dramatically during phase transfer of the various metal salts, this observation is consistent with mixed Au—Cu containing TOAB micelles prior to NaBH₄ reduction.

On the other hand, no difference in ¹H line broadening is observed in reaction mixtures containing Co(II), which is consistent with observations from the one-phase synthesis where no redox chemistry occurs between the thiol functionality and the Co(II) metal center. However, a color change from orange to blue is noted in the reaction mixture containing both Au and Co upon DDT addition, indicating that the Au(III) species has become colorless leaving only the blue Co(II) observable, consistent with a reduction of Au(III) to Au(I). Therefore, in the ¹H NMR spectra containing DDT, we assign the Au and Cu to be in the +1 oxidation state and the Co to be in the +2 oxidation state.

The spectroscopic observations indicate that metal halide anions bind to the surface of the inverse TOA⁺ micelle. Indeed, spatial segregation between the DDT and the metal precursors is suggested by the lack of further oxidation products beyond the dodecyl disulfide (DDS) in the 1D ¹H NMR. Further analysis using PFG ¹H NMR shows that in all cases, no high molecular weight metal-ligand complexes are detected in any of the reaction mixtures. In addition, no evidence of a precipitate was observed in any of the reaction mixtures pre-NaBH₄ addition after a few days of aging, indicating that no metal-thiolate polymeric species had formed. The lack of metal-sulfur bonds in a typical Brust-Schiffrin synthesis, but rather a TOA⁺-[M(I)X]⁻ precursor species is consistent with previous reports from other groups. This finding suggests that metal-sulfur bond attachment occurs after NaBH₄ addition in two-phase syntheses, once the particle grows large enough to reach the aqueous-organic interface, providing an alternate formation pathway compared to one-phase approaches.

Final Particle Characterization.

Pure AuNPs as well as bimetallic AuₓCu_{y} and AuₓCo_{y}NPs were synthesized using both the one phase and two phase synthetic methods to investigate the influence of metal-thiolate vs. TOA⁺-[M(I)X]⁻ precursor on final product. Both metal core and hydrodynamic size of the resulting AuₓTM_{y}NPs was characterized by both HRTEM and PFG ¹H NMR techniques, respectively. The results for all compositions and preparation methods are consistent with the formation of a 1-3 nm diameter metal core. The final nanoparticle composition was characterized by ICP-MS, STEM-EDS point spectra, HRTEM lattice analysis, XPS, and Auger electron spectroscopy.

Further analysis of a moderate Cu incorporation for both the one- (20% Cu) and two-phase (12% Cu) AuₓCu_{y}NPs showed differences in the spatial composition of the NP, based on preparation method (Table 2). Based on this data, the one phase AuₓCu_{y}NPs show a small amount of Au—Cu bonding as evidenced by the presence of these bonds from both the Au L3 edge (Au—Cu coordination number, CN_{Au-Cu}=0.20) and the Cu K edge (CN_{Cu-Au}=0.81). In addition, both Au—S and Cu—S bond lengths, 2.327 and 2.298 Å respectively, indicate that both elements are present at the particle surface.

In contrast to the one phase AuₓCu_{y}NPs, the two phase particles show the presence of only Cu at the particle surface as indicated by the relatively short Cu—S bond of 2.276 Å. The longer Au—S bond of 2.433 Å and the higher Au—Au coordination number indicates that Au is one layer below the Cu-based, thiolated surface. In order to determine if a Au core-Cu thiolate shell morphology was feasible, XAS data for 100% Au controls for both preparation methods were collected. In these cases, Au would inevitably be present on the particle surface and would be expected to participate in a Au-thiolate bond. The XAS data for both the one-phase and the two-phase AuNP syntheses are consistent with the formation of a small gold nanoparticle core capped with Au-thiolate units with Au—S bond lengths of 2.338 Å and 2.344 Å, respectively.

Additionally, the differences in morphology observed in XAS measurements are consistent with the findings from XPS and AES as a function of various Au—Cu composition. In all one phase AuₓCu_{y}NPs, a binding energy shift due to the formation of metallic Au—Cu bonds is observed as a function of composition. Further, chemical state analysis with AES is consistent with the formation of an alloy and the presence of both elements at the particle surface.

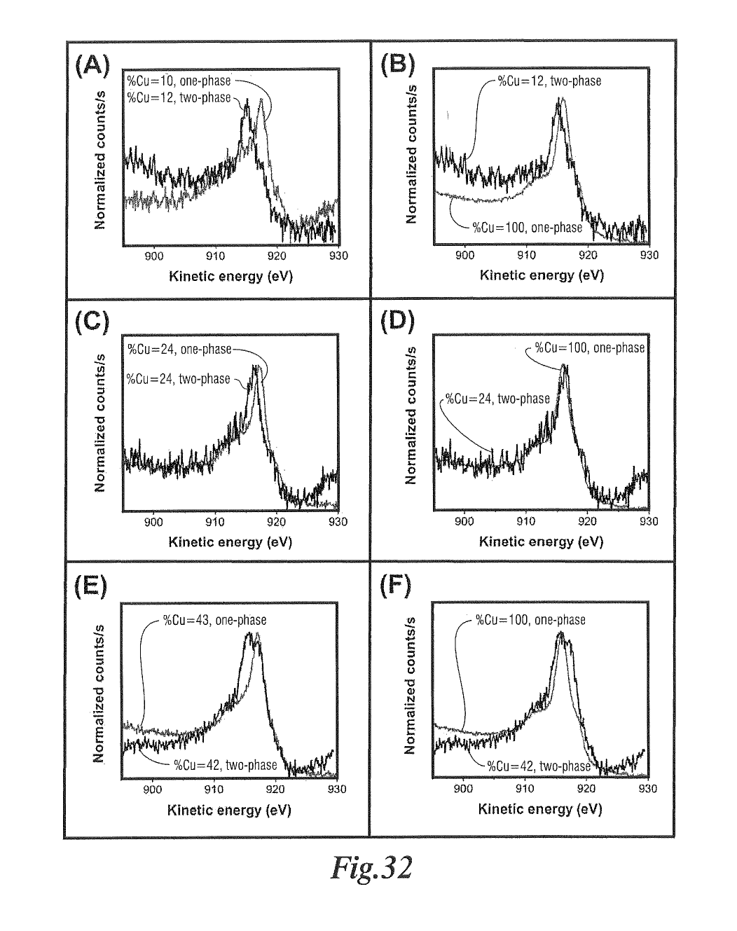

Consistent with the XAS, further analysis with XPS and AES indicates that at low % Cu, all Cu is present in the ligand layer as Cu—S. As the amount of Cu is increased above 30% Cu atoms would begin to migrate into the particle interior. Since diameter remains relatively constant between syntheses, only ˜30 metal atoms are thought to participate in a typical staple motif for a M₁₄₄ particle. In the AES spectra of 50% Cu incorporation in a two phase particle, two distinct peaks can be observed: one for Cu—S and one for Cu that is consistent with metallic Cu that is phase separated from Au. See FIGS. 31 and 32.

In addition to X-ray analysis, differences were observed in the HRTEM lattice analysis that were consistent with phase segregation for AuₓCu_{y}NPs prepared via a two-phase approach, despite the fact that STEM-EDS analysis indicated that both Au and Cu were co-localized in the same NP. These particles showed distorted lattices and FFTs, with some FFTs containing multiple spots, while AuₓCu_{y}NPs prepared via a one phase method exhibited uniform lattices and FFTs. Further, the two preparation methods yielded particles that exhibited distinct optical properties. One-phase AuₓCu_{y}NPs display a relatively featureless absorption spectra, yet all particles exhibit an excitation peak at λₘₐₓ ˜360 nm. When excited at 360 nm, the particles emit NIR PL with a λₘₐₓ that is tunable based on %

TABLE 2

| Sample | Path | CN | R (Å) | σ² (Å²) | ΔE₀ (eV) |
|---|---|---|---|---|---|
| Au-PEGSH | Au—S | 0.58 | 2.338 | 0.00073 | −0.3 |
| capped NPs | Au—Au | 7.19 | 2.799 | 0.0157 | −0.3 |
| AuₓCu_{y}- | Au—S | 1.13 | 2.327 | 0.0039 | −1.1 |
| PEGSH | Au—Au | 3.59 | 2.740 | 0.0132 | −1.1 |
| capped NPs | Au—Cu | 0.20 | 2.752 | 0.0032 | −1.1 |
| (y = 20%) | Cu—S | 1.62 | 2.298 | 0.0037 | 2.7 |
| | Cu—Au | 0.81 | 2.812 | 0.0117 | 2.3 |
| Au-DDT | Au—S | 0.90 | 2.344 | 0.0035 | 1.9 |
| capped NPs | Au—Au | 5.82 | 2.857 | 0.0067 | 1.9 |
| AuₓCu_{y}-DDT | Au—S | 0.89 | 2.433 | 0.005 | 0.0 |
| capped NPs | Au—Au | 6.63 | 2.813 | 0.0078 | 0.0 |
| (y = 12% Cu) | Cu—S | 2.68 | 2.276 | 0.0076 | 1.8 |
| | Cu—Au | 0.60 | 2.754 | 0.0021 | 0.5 |

Cu composition and ranges from 939-1096 nm. AuₓCu_{y}NPs synthesized using the original Brust-Schiffrin two phase approach do not exhibit detectable photoemission in the near infrared or the visible region when excited at any wavelength. Their extinction spectra also shows a broad feature at ˜500 nm, characteristic of small gold-containing nanoparticles, suggesting differences in the optoelectronic properties between the two particle types.

The morphological differences in composition are also consistent with the precursor chemistry that was observed prior to NaBH₄ addition, and provide crucial insight to explain the observations in Au—Co systems. In the Au—Cu one-phase synthesis, mixed metal-thiolate trimers and tetramers are formed prior to BH₄ reduction. Recent theoretical work on NaBH₄ reduction of Au₄(SR)₄ structures suggests that hydride addition results in reduction of the overall pre-nucleation species by removing a ligand, producing Au₄(SR)₃⁻ and forming the initial Au—Au bonds. Further, this work also demonstrated that electron addition to Au₄(SR)₄ to produce Au(0) monomers was energetically unfavorable, indicating that this pathway was unlikely the route to nanoparticle formation when these species were present. We postulate that in one-phase syntheses, the metal with the highest reduction potential is reduced first, and the metal-thiolate reduction occurs later due to the lowered reduction potential and proceeds via hydride addition. In addition, we hypothesize that, consistent with theoretical studies, the pre-nucleation structure would be reduced as a whole via hydride addition and metal-metal bonds would be formed from the structure itself, rather than losing a M⁰ atom. Since one-phase Au—Cu syntheses exhibit Au—Cu-thiolates prior to NaBH₄ addition, this reduction pathway would allow for both Au and Cu to be present on the particle surface, as indicated by X-ray data.

For Au—Cu two-phase syntheses, metal halide anions are coordinated to the TOA⁺ surface and are segregated from the thiolated protecting ligand by the inverse micelle-like architecture. Therefore, when NaBH₄ is injected, nucleation would proceed based on standard reduction potentials of the metal complexes (for comparison, E⁰ for [AuCl₂]⁻=+1.15 V and [CuCl₂]⁻=+0.19 V). Therefore, Au would nucleate first because of the higher reduction potential, forming the nanoparticle core. When Cu is reduced, it is likely readily re-oxidized to form the passivating bonds with the thiolated capping ligand. This formation mechanism leads to a bimetallic Au—Cu nanoparticle with a Au core capped with Cu—S passivating units at low Cu concentrations, as indicated by XAS and XPS. As Cu concentration is increased, the particle remains capped by Cu—S units, but some Cu intercalates the Au core, as shown by XPS.

In addition, the postulated two-phase and one-phase formation mechanisms can be extended to understand the incorporation behavior in the Au—Co system. For two-phase syntheses, the nucleation event is dominated by the reduction potential of the metal precursors present in the inverse micelles. Again, the reduction potential of the Au precursor remains much higher than that of the Co, and is likely reduced first. However, when Co is reduced and added to the particle surface, unfavorable Co—S bonds and Co surface energies likely lead to Co being expelled from the particle altogether, resulting in no Co incorporation in two-phase preparations.

On the other hand, one phase approaches show continuous Co incorporation once the initial molar ratio of Co is ≧50%. At lower initial molar ratios of Co, NMR and XAS indicate that a small amount of Au(0) forms prior to NaBH₄ addition, probably forming the initial nuclei for nanoparticle growth. Similar to the two-phase synthesis, if a Au core begins to grow and Co nucleates on top, no Co is incorporated in the final particle. However, if Au(0) formation is prevented and all of the Au precursor in solution is present as an Au-thiolate tetramer, the Co and Au precursors have the opportunity to nucleate at a similar point in time, allowing incorporation into the same nanoparticle, consistent with characterization by STEM-EDS, XPS, and ICP-MS. Previous electrochemical studies have found that the reduction potential of Au(I)-thiolates with a cysteinato ligand are substantially lower than their Au(III) counterparts at E⁰=−0.14 V, much closer to that of Co(II) at E⁰=−0.28 V. Other studies have shown the importance of reduction potential matching to achieve binary metal mixing, indicating that this condition is necessary to incorporate both Au and Co into a single architecture, despite the fact that they do not coexist in the same pre-nucleation species.

Overall, a detailed characterization of the mono- and bimetallic precursors present in both one-phase and two-phase noble metal nanoparticle alloy syntheses has been provided. Observed was that one phase syntheses form metal-thiolate trimers and tetramers, allowing the synthesis of mixed-metal architectures of both bulk miscible (Au—Cu) and bulk immiscible (Au—Co) elements. Mixed-metal structures were achieved via co-reduction in one-phase syntheses, either through incorporation into the same pre-nucleation species, as in the case of Au—Cu, or reduction potential matching, in the case of Au—Co. In addition, two-phase syntheses contain metal halide anions coordinated to the TOA⁺ quaternary ammonium surface, likely confined inside an inverse micelle-like architecture and isolated from the thiolated capping ligand. Here, because individual metal reduction occurs independently, transition metal incorporation is based on reduction rate of the precursor species. Overall, we find that both methods incorporate transition metals into the Au nanoparticle based on reduction potential of the constituent precursors. Yet, structural data suggests that final atom position can be controlled by manipulating the composition of the precursor chemistry prior to NaBH₄ addition.

### Gold-Cobalt Nanoparticle Alloys Exhibiting Tunable Compositions, Near-Infrared Emission, and High T₂ Relaxivity

In a preferred embodiment of the present disclosure, synthesis of discrete, composition-tunable gold-cobalt nanoparticle alloys (% Co=0-100%; diameter=2-3 nm), in contrast with bulk behavior, which shows immiscibility of Au and Co at room temperature across all composition space has been demonstrated. These particles are characterized by transmission electron microscopy and ¹H NMR techniques, as well as inductively coupled plasma mass spectrometry, X-ray photoelectron spectroscopy, and photoluminescence spectroscopy. In particular, ¹H NMR methods allow the simultaneous evaluation of composition-tunable magnetic properties as well as molecular characterization of the colloid, including ligand environment and hydrodynamic diameter. These experiments also demonstrate a route to optimize bimodal imaging modalities, where we identify AuₓCo_{y}NP compositions that exhibit both bright NIR emission (2884 M⁻¹cm⁻¹) as well as some of the highest per-particle T₂ relaxivities (12200 mM_{NP}⁻¹s⁻¹) reported to date for this particle size range.

The now canonical relationship between nanoparticle morphology and nanoparticle physical properties is remarkable and continues to produce an inspiring suite of new materials, physical insights, and technological capabilities. In the case of metallic nanoparticles, the majority of these advances have been made with particles comprised of a single element. Yet, centuries of metallurgy indicate that a vast new dimension of particle properties and applications may emerge with the creation of alloyed nanoparticle colloids. Further, in applications with narrow tolerance for particle dimensions and/or surface chemistry (e.g., biologic or catalytic applications) accessing a diversity of nanoparticle behaviors from a single composition is challenging. To address this challenge, a variety of multimetallic nanoparticles have been synthesized including core-shell, hollow, Janus, and alloyed morphologies.

One attractive class of alloys is the combination of noble metals with more earth-abundant transition metals. These metal mixtures have generated considerable interest for cost reduction and/or performance enhancement of precious metal catalysts as well as for stabilization (e.g., from oxidation) of ferromagnetic elements such as Fe and Co in materials for data storage and theranostic applications. Optical properties can also be enhanced via alloying. For example, we have reported the composition-tunable near-infrared (NIR) photoluminescence (PL) properties of gold-copper (AuₓCu_{y}) nanoparticle alloys (diameter, d=2-3 nm) Combining the optical features of Au with ferromagnetic (in the bulk) elements such as Ni, Co, or Fe is an opportunity to leverage several of these effects within a single particle architecture.

However, bulk phase diagrams indicate that Au is largely immiscible with each of these metals at temperatures below 400° C. In the case of cobalt, the immiscible behavior is dramatic, with no miscibility or intermetallic states predicted below 400° C. across all composition space. Likewise, simulations for surface alloys of Au and Co consistently predict segregation behavior for both Au host-Co solute and Co host-Au solute surfaces. Yet, some reports indicate that materials at the nanometer length scale may deviate significantly from these trends. At particle sizes between 95-2590 atoms, Nørskov and co-workers have reported that particle size alone can influence metal segregation behaviors. More recently, Schaak and co-workers have developed a spectrum of preparations for the formation of nanocrystalline alloyed materials, which are analyzed to be representative of L1₂ intermetallic states. In particular, the authors use metal diffusion at 250° C. to create Au₃Ni, Au₃Fe, and Au₃ Co particles with dimensions ranging from ˜10-30 nm. Interestingly, these intermetallics are not predicted by bulk phase diagrams, and instead were one of the first indications that nanoscale colloids may form a greater diversity of alloyed architectures than has previously been observed in the bulk.

In a preferred embodiment of the present disclosure, a combination of rapid metal ion reduction and surface chemistry-based strategies are used to form small (d=2-3 nm), discrete, composition-tunable gold-cobalt nanoparticle (AuₓCo_{y}NP) alloys at room temperature in water. This approach produces AuₓCo_{y}NPs across a wide range of compositions (0 to 100% Co) and indicates a new pathway to synthesize these previously inaccessible alloys. The resulting particles exhibit composition-tunable magnetic susceptibility as well as some of the highest reported values for T₂ relaxivity as compared to super-paramagnetic iron oxide nanoparticles (SPIONs) in a similar size range. At the same time, the particles retain attractive optical features associated with Au at this length scale, specifically, bright NIR emission. Tuning composition, we then identify optimum architectures for bimodal imaging properties, while maintaining particle size and surface chemistry.

TABLE 3
Size, composition, photoluminescence, and magnetic property analysis of AuₓCo_{y}NPs.

| Initial | | | | | | | | | |
|---|---|---|---|---|---|---|---|---|---|
| molar | NP | | | NP size | ε at | | | | r₂ |
| ratio | composition | Lattice | NP size | (nm) | 360 nm | | | Magnetic | (mM_{Co}⁻¹s⁻¹/ |
| added | (% Co) | constant (Å) | (nm) | PFGSE- | (×10⁵ | Φ | Brightness | susceptibility | mM_{NP}⁻¹s⁻¹) |
| (% Co) | ICP-MS | HRTEM | HRTEM | NMR | M⁻¹cm⁻¹) | (×10⁻³) | (M⁻¹cm⁻¹) | (×10⁻⁶ cm³g_{NPs}⁻¹) | 7 T |
| 0 | 0 ± 0 | 3.96 ± 0.05 | 2.2 ± 0.5 | 4.1 ± 0.1 | 9.3 ± 2.3 | 0.40 ± 0.02 | 374 | −0.65 ± 0 | NA |
| 50 | 1.6 ± 0.1 | 3.85 ± 0.03 | 2.3 ± 0.5 | 4.2 ± 0.1 | 12.6 ± 4.8 | 2.29 ± 0.49 | 2884 | −0.39 ± 0.04 | NA |
| 60 | 7.7 ± 0.7 | 3.70 ± 0.03 | 2.2 ± 0.2 | 4.3 ± 0.3 | 8.7 ± 1.2 | 2.80 ± 0.64 | 2430 | −0.20 ± 0.05 | 1.5/49 |
| 70 | 26.8 ± 2.0 | 3.75 ± 0.04 | 2.3 ± 0.5 | 4.3 ± 0.1 | 4.6 ± 1.1 | 3.00 ± 0.15 | 1373 | 0.55 ± 0.34 | 2.4/209 |
| 80 | 48.1 ± 2.7 | 3.73 ± 0.03 | 2.2 ± 0.3 | 4.1 ± 0.3 | 9.2 ± 1.8 | 2.52 ± 0.36 | 2322 | 3.24 ± 0.96 | 6.8/1750 |
| 85 | 62.0 ± 2.0 | 3.88 ± 0.05 | 2.1 ± 0.2 | 4.3 ± 0.4 | 6.1 ± 0.9 | 0.50 ± 0.26 | 305 | 5.34 ± 1.01 | 11/3650 |
| 90 | 80.7 ± 2.5 | 3.90 ± 0.05 | 2.2 ± 0.4 | 4.3 ± 0.1 | 7.1 ± 0.1 | 0.30 ± 0.16 | 211 | 8.51 ± 1.23 | NA |
| 100 | 100 ± 0 | 4.79 ± 0.05 | 2.9 ± 0.5 | 4.9 ± 0.1 | NA | NA | NA | 11.26 ± 1.34 | 26/12200 |

*All reported values are the average of at least 3 independently synthesized trials. The values for NP size are reported with the standard deviation of the mean. All other values are reported with the standard error.

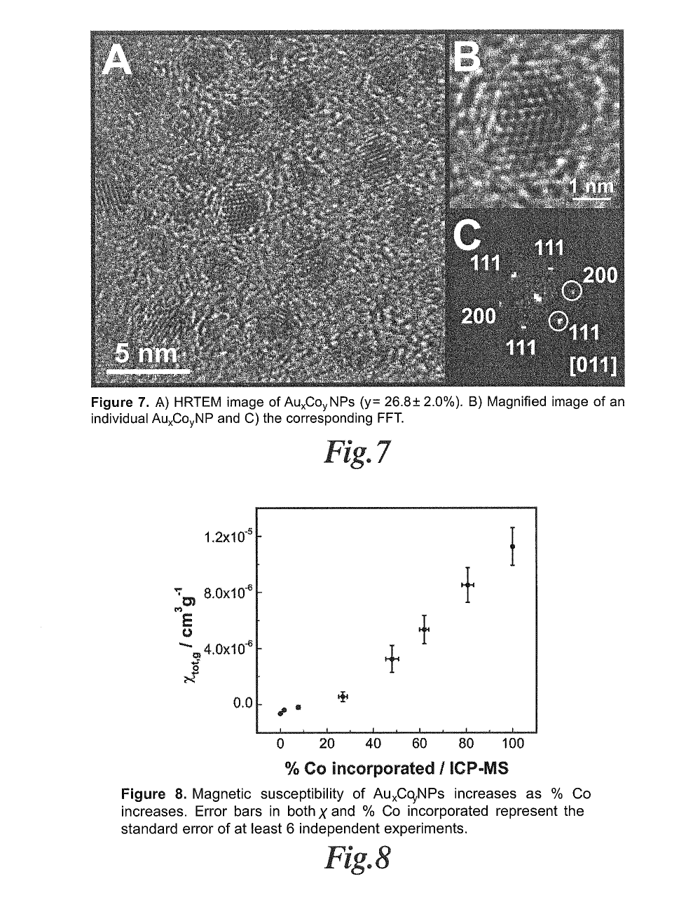

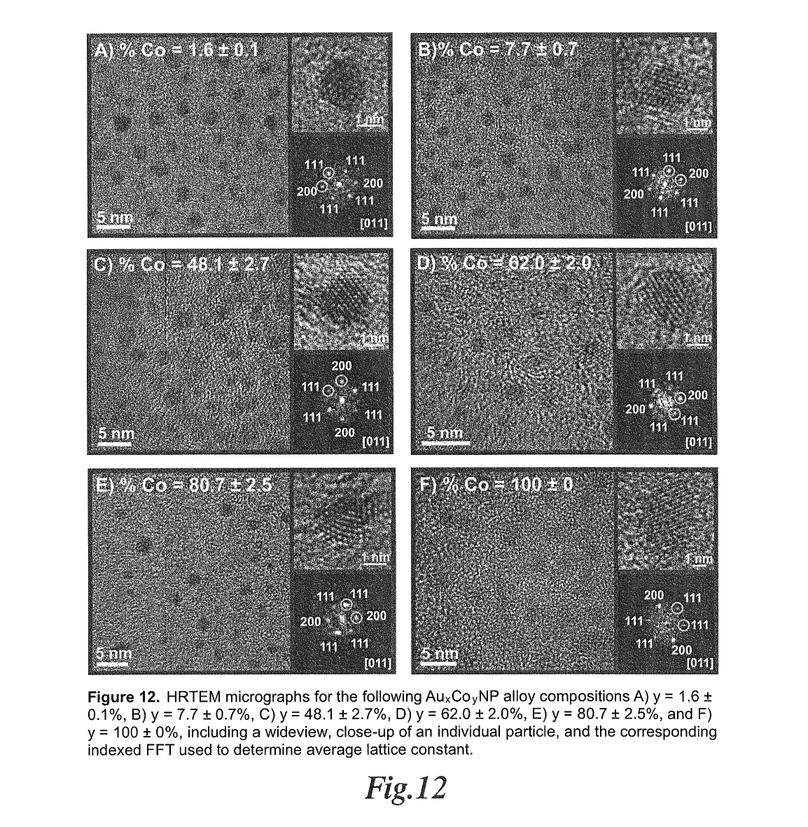

In a typical experiment, AuₓCo_{y}NP alloys were synthesized by co-reduction of HAuCl₄ and Co(NO₃)₂ with NaBH₄ at room temperature in an aqueous solution containing the capping ligand, poly(ethylene glycol) methyl ether thiol (PEGSH, average Mₙ=1000 Da). NaBH₄ is an attractive reducing agent because it is water soluble, can reduce both metal precursors, and in pure metal nanoparticle syntheses (e.g. Au and Ag), the oxidized byproducts are not known to influence the reaction. We choose a thiolated ligand, because they are associated with the synthesis of small, stable Au nanoparticles. A PEG moiety is chosen for water solubility and biocompatibility. The initial molar ratio of Co to Au was varied from 0-100% Co, while maintaining the same total metal, capping ligand, and reducing agent concentrations (complete synthesis details are included in Experimental and Supporting Information (SI) sections). All nanoparticle products were characterized using UV-visible spectroscopy, inductively coupled plasma mass spectrometry (ICP-MS), X-ray photoelectron spectroscopy (XPS), photoluminescence spectroscopy, transmission electron microscopy (TEM), and ¹H nuclear magnetic resonance (NMR) techniques. Figures of merit from these studies are listed in Table 3. FIG. 7 shows high-resolution transmission electron microscopy (HRTEM) images of AuₓCo_{y}NPs (x=100%-y; y=26.8±2.0% Co as measured by ICP-MS; see FIG. 12 for HRTEM of additional AuₓCo_{y}NP compositions). In all cases, AuₓCo_{y}NPs are observed as pseudospherical, discrete, and crystalline nanoparticles with average metallic core diameters between 2.1-2.3 nm and a standard deviation of <20% (FIG. 7 and FIG. 12). The hydrodynamic diameter of the AuₓCo_{y}NPs was calculated from the diffusion coefficient as measured by pulsed-field gradient stimulated echo (PFGSE) ¹H NMR. The hydrodynamic diameters of all AuₓCo_{y}NPs are 4.1-4.3 nm, consistent with a 2.1-2.3 nm metallic core diameter capped with a monolayer of random coil PEGSH (M n=1000 Da).

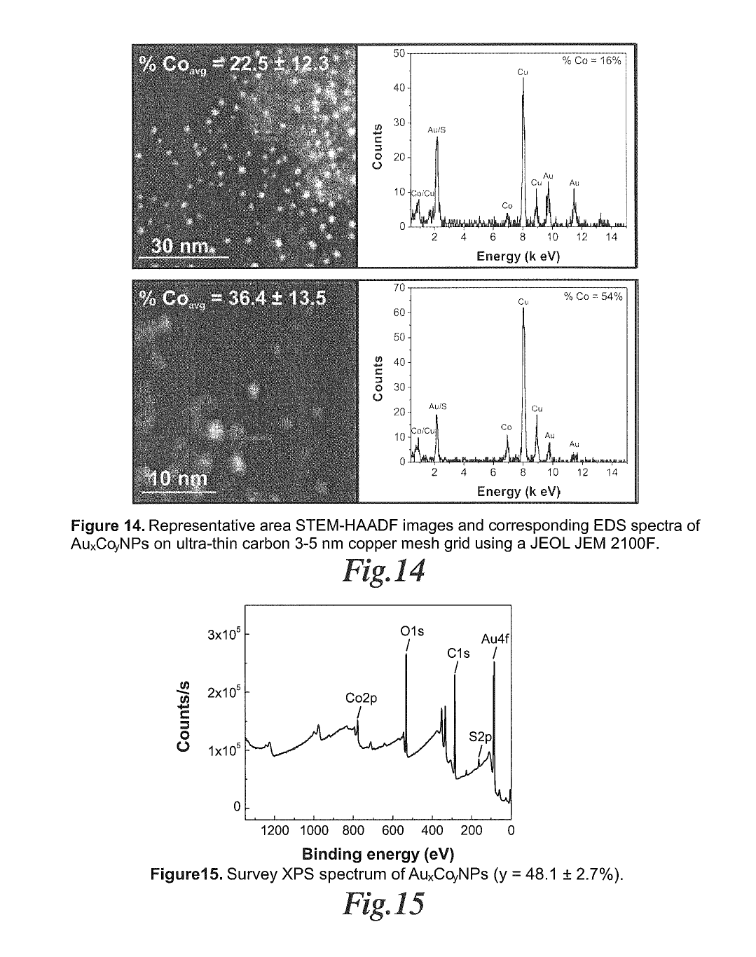

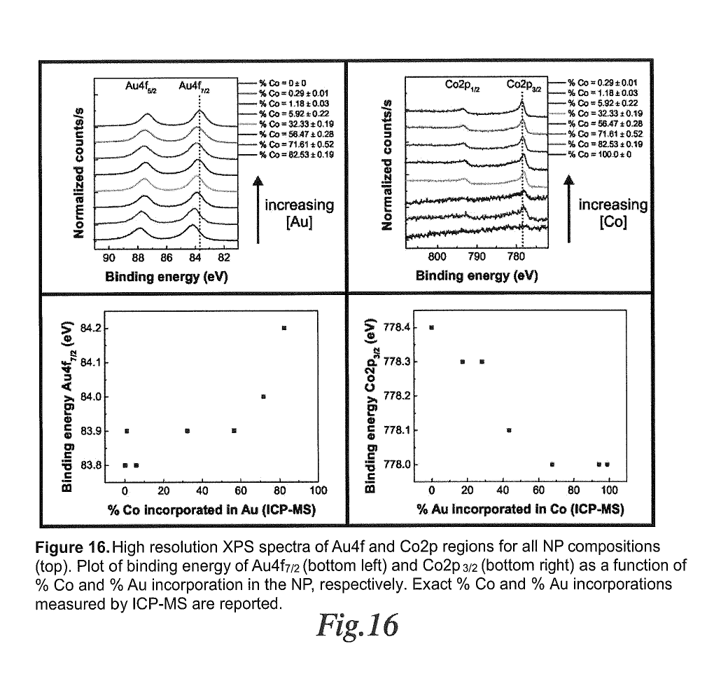

To assign the composition and composition morphology of the resulting particles, we use a combination of several techniques. First, we analyze particle crystallographic features using HRTEM. The bulk lattice constant of Au_{fcc,} a=4.079 Å and the bulk lattice constant of metallic Co_{hcp}, a=2.503 Å, c=4.061 Å or Co_{fcc,} a=3.545 Å. Therefore, regardless of the overall crystal system adopted by the particle, as % Co increases, the particle lattice constant(s) are expected to decrease with respect to either bulk Au or the lattice constant of a pure Au particle of this size (100% AuNPs=3.96 Å, Table 3). Initially, our results follow this trend where increasing Co incorporation leads to a decrease in observed particle lattice constants (Table 3). However, as the % Co incorporation reaches a threshold (>60%), the observed lattice constants begin to increase. This increase is likely due to the formation of a cobalt oxide, which may be expected since our synthesis is conducted in air and in water (this assignment is supported by XPS analysis, vide infra and FIG. 16). Importantly, no core-shell architectures are observed in either HRTEM or scanning transmission electron microscopy (STEM) analysis (FIG. 14), and the distribution of lattice constants is not bimodal, indicating that there are not two populations of particles each comprised of only one metal.

After analysis of lattice features and general morphology, we use three techniques to analyze elemental composition. ICP-MS and XPS were used to evaluate the metal atom concentrations and oxidation states of the bulk colloid, respectively. STEM-EDS point spectra were used to assess the composition of individual particles (FIGS. 14, 18 and Tables 5, 6). ICP-MS analysis indicates that little to no Co incorporation is observed until the initial molar ratio of Co was increased to 50%. At initial molar ratios above 50% Co, the nanoparticles exhibit a continuously tunable stoichiometry, and the final incorporation of Co into the Au nanoparticles was varied from 1.6-89.8% (FIG. 18 and Table 3). The initial lag in Co incorporation may be a product of the disparity in reduction potential between Co(II) and Au(III) species which results in less available Co monomer (here, referring to “monomer” as described by LaMer) at the critical concentration for homogeneous nucleation of the particle solid phase. Previous reports indicate that co-reduction during nucleation was a crucial factor in the formation of intermetallics and larger alloyed shells. Differences in reduction potential are also thought to play a large role in the formation of coreshell particles or incomplete mixing of the two components (e.g., heterogeneous solid solution or “island” formation). We hypothesize that above 50% initial molar ratio of Co, a threshold amount of Co monomer is available to co-nucleate with Au monomer, allowing both elements to be incorporated into a single particle.

To analyze the composition of individual particles, we use STEM-EDS point spectra. For a sample of nanoparticles synthesized with a given molar ratio of Au:Co, individual particle compositions were measured by EDS, and spectra were obtained from several different particles to establish an average particle composition. Average compositions agreed well between ICP-MS and STEM-EDS analysis. However, it is important to note that using STEM-EDS, we observed that particle-to-particle composition was more heterogeneous as % Co increased, and this heterogeneity is consistent with the increased variation for the same initial molar ratios as measured by ICP-MS (i.e., the standard error for composition increases with increasing % Co, Table 3 and FIG. 18). Particle-to-particle composition heterogeneity may be a result of our synthetic strategy. For example, the rapid particle nucleation approach can be viewed as an analog to the bulk diffusion-quench processes used to form bulk alloys. In diffusion-quench methods, a given ratio of two metals are heated together and entropy drives metal mixing. The mixture is then cooled to “freeze” the combined state. In our synthesis, instead of cooling, we rapidly increase the solution saturation in metal precursor, which induces nucleation of the solid phase. During this step, there may be limited selectivity for metal incorporation into the particle. Instead, we hypothesize that the local molar ratio of metal precursor in solution determines the ratio of the two metals incorporated into the final nanoparticle architecture. It is important to note that comparison of XRD spectra to determine particle composition was not possible from particles of this size range due to significant line broadening, which is consistent with mathematical predictions of X-ray optics.

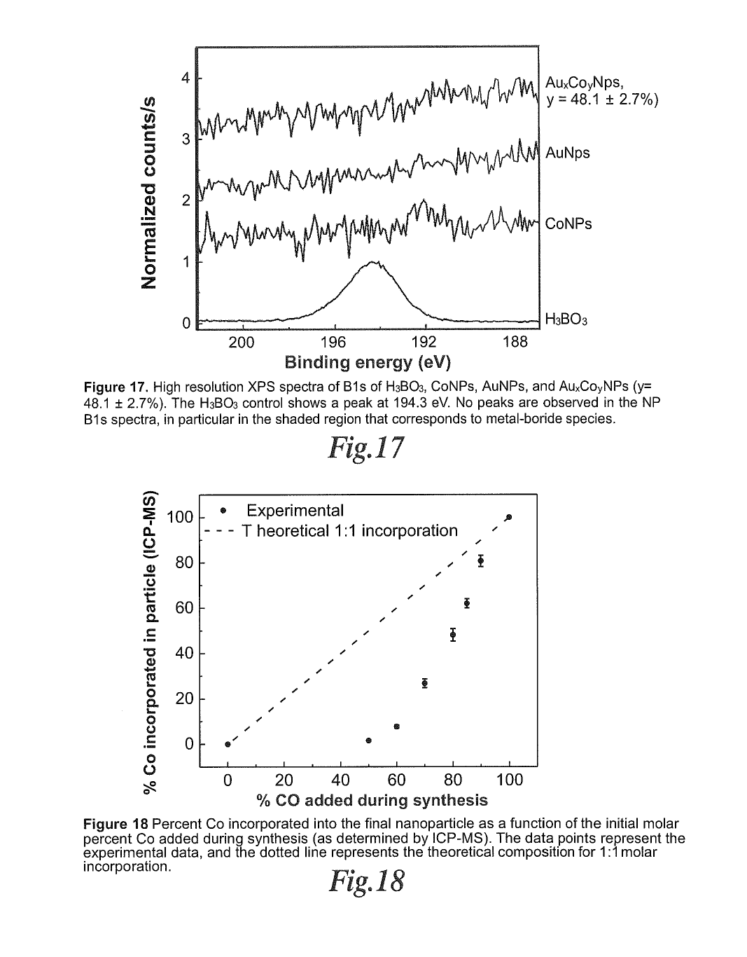

To further characterize the composition and oxidation state of the AuₓCo_{y}NP alloys, all particle compositions were analyzed by XPS (FIGS. 15 and 16). Survey spectra showed the presence of Au, Co, C, O, and S in all samples (with the exception of Au₁₀₀NPs and Co₁₀₀NPs, which lacked Co and Au peaks, respectively). Previous syntheses using pure Co precursor under similar reaction conditions have also observed boron in the particle products, however we do not observe boron signal in any XPS spectra (FIG. 17), which indicates that borohydride, borate byproducts or cobalt-boride materials are not present in the purified final nanoparticle products. A shift of the Au4f_{7/2} peak from Au₁₀₀NPs at 83.8 eV to higher energy is observed with increasing % Co incorporation, suggesting a continuous change in the Au environment that is consistent with alloy formation. Analysis of the Co2p_{3/2} peak shows the presence of metallic cobalt as a sharp, narrow band with binding energies ranging from 778.0-778.4 eV, in all cases. From a pure Co phase to an alloyed phase, we observe a shift to lower binding energy of the Co2p features. From a pure Au phase to an alloyed phase, we observe a shift to higher binding energy of the Au4f peaks. These binding energy shifts do not follow trends expected from electronegativity arguments, but instead are consistent with electron density moving from Au to Co. Similar trends have been observed for other Au-transition metal alloys, such as Au—Ni, where Ni2p_{3/2} binding energies decrease and Au4f_{7/2} binding energies increase when comparing the pure metal phase to an alloyed composition. For high concentrations of Co (>60% Co incorporation) a shoulder is present at ≈781 eV. This binding energy region is consistent with Co(II) or Co(III) species. However, no corresponding satellite peaks are observed (≈786 eV), which indicates that where oxidation is present, the concentration is low (Figure S6). Limited oxidation of the Co, despite a synthesis conducted in air and water, is consistent with stabilizing trends observed in other noble-transition metal alloys such as PtFe and PtCo, where the first row transition metal exhibits enhanced resistance to oxidation when alloyed with a more noble counterpart.

Next, we analyze particle magnetic properties and also use this analysis as an additional metric to assess composition tunability. In order to determine the magnetism of AuₓCo_{y}NPs, we have used the Evans' method to measure the mass magnetic susceptibility at room temperature. Here, the Evans' method is an alternative to superconducting quantum interference device (SQUID) analysis, which requires significantly more material, especially for small particle sizes where diamagnetic capping ligands can quench the magnetism of surface atoms, which are a large percentage of total atoms in the sample (˜40% for d=2.2 nm). Using the Evans' approach, we analyzed a series of AuₓCo_{y}NP compositions (0-100% Co incorporation with 5% initial molar ratio step sizes, FIG. 8 and FIG. 20), to determine the relationship between particle composition and particle susceptibility. Here, we found that by controlling the % Co incorporated in the final AuₓCo_{y}NPs we could achieve continuously tunable magnetic susceptibility from −0.39×10−6 to 11.26×10⁻⁶ cm³g_{NPs}⁻¹. The reported values represent the total mass magnetic susceptibility of the sample, which is comprised of both the diamagnetic and paramagnetic contributions (Table 3). The magnetic susceptibility values reported here, as well as relaxivity measurements discussed below, are consistent with previous reports of a variety of superparamagnetic nanoparticles, including AuNi nanoparticles and SPIONs.

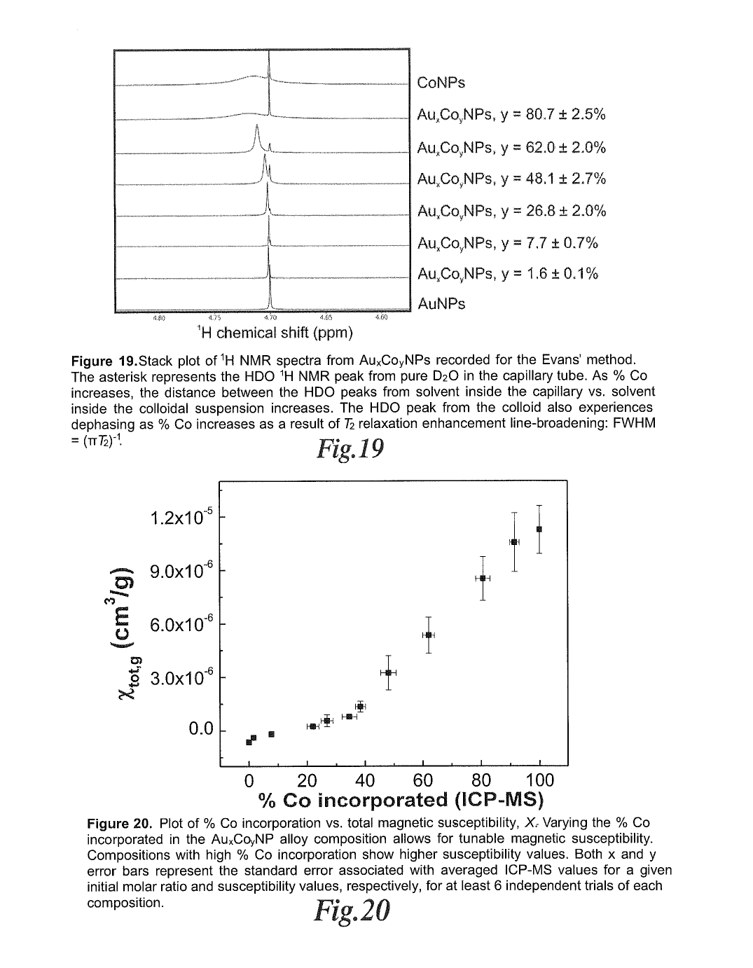

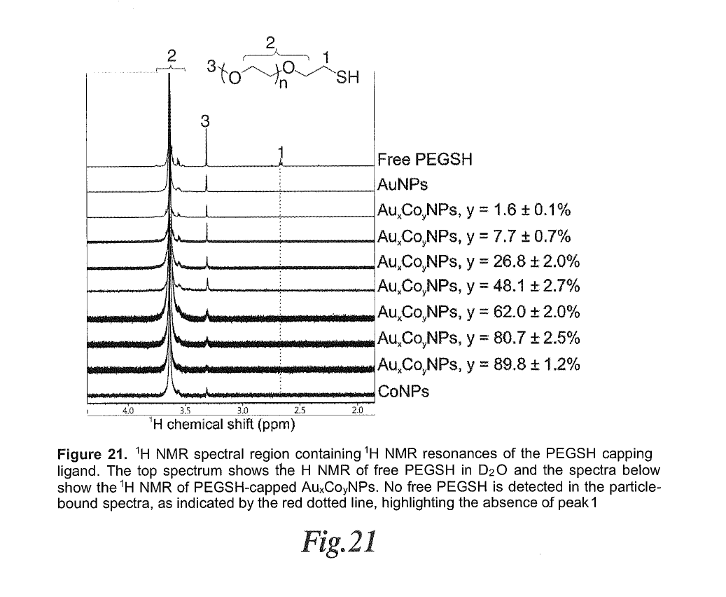

By using a molecular characterization method to analyze our magnetic susceptibility, we were also able to directly observe the ¹H NMR spectrum of the NP ligand shell in each sample within a single experiment (1D ¹H NMR spectra in FIG. 19). Here, ¹H NMR spectra show an absence of the thiol proton as well as the directly adjacent CH₂ protons on the PEGSH (FIG. 21). The absence of these peaks from the ¹H NMR spectra is consistent with significant dephasing, which is expected to be a result of a chemical shift distribution from various PEGSH binding sites as well as conduction electrons at the NP surface. Control experiments were performed to ensure that changes in magnetic susceptibility and relaxivity were not the result of excess reactant impurities (see FIG. 21 and corresponding discussion).

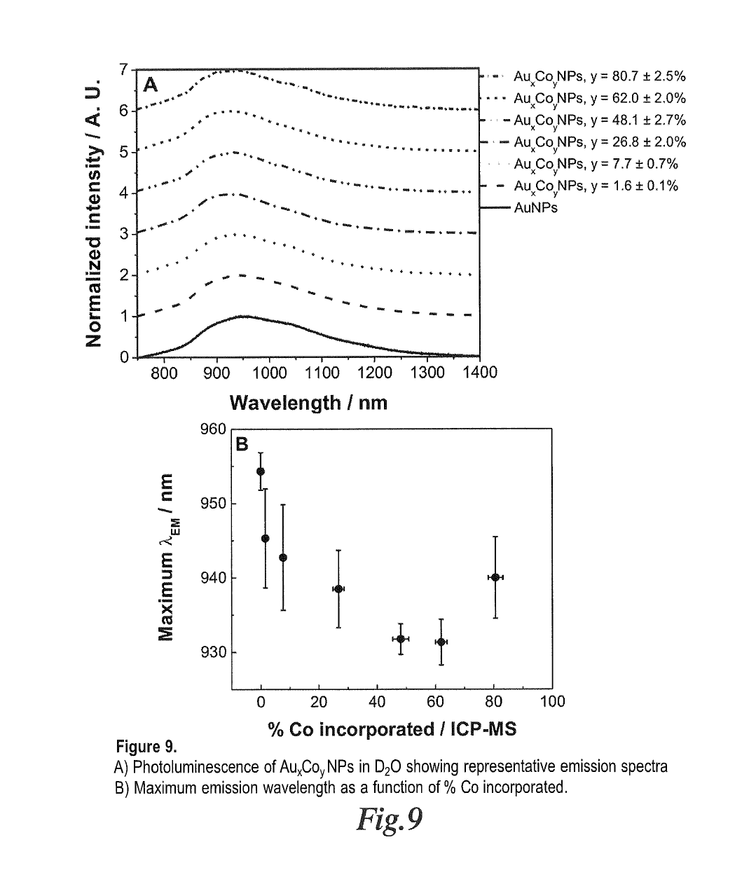

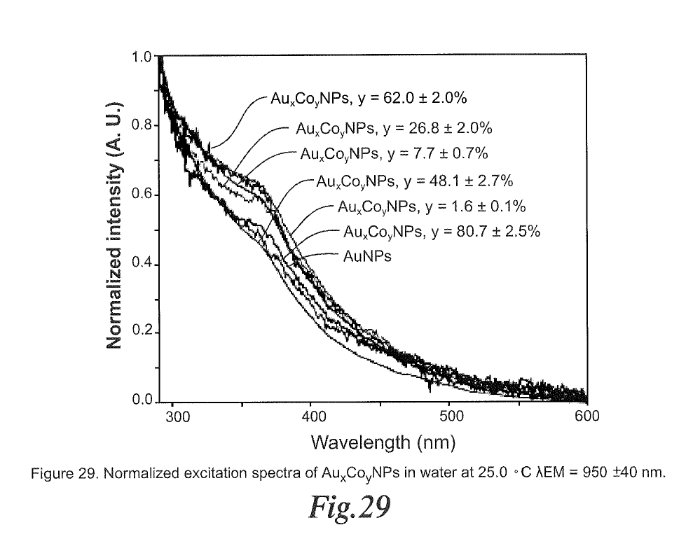

Remarkably, AuₓCo_{y}NPs also exhibit PL in the NIR spectral region, which to the best of our knowledge, is the first observation of PL from Au—Co alloys at any length scale. Here, all compositions of the AuₓCo_{y}NPs exhibit NIR PL, with the exception of 100% CoNPs (Table 3). Excitation spectra from these particles are consistent with previous excitation spectra obtained for Au and AuₓCu_{y} NPs (FIG. 29). Interestingly, in the case of AuₓCo_{y}NPs, a hypsochromic shift (≈25 nm) in the maximum emission wavelength relative to 100% AuNPs is observed (FIG. 9) with increasing % Co incorporation. This trend is observed until Co concentration in the nanoparticle reaches >60% incorporation. Beyond this concentration, the maximum emission wavelength exhibits a bathochromic shift toward the emission maximum from 100% AuNPs. This % Co composition is also coincident with our observation of increases in Co oxidation via XPS, as well as increases in lattice constants.

Previous work indicates that the NIR emission originates from a surface charge-transfer state comprised of Au-thiolate interactions. In the case of the AuₓCu_{y}NPs, we hypothesized that the presence of Cu in the surface region (surface or subsurface layers) changes the energy of this Au-thiolate interaction possibly by replacing one or more of the bonding Au atoms with a Cu atom, consistent with previous reports. The presence of PL from the Au x Co y NPs, but less dramatic composition dependence of the maximum λ_{EM} (FIG. 9), indicates that the incorporation of Co into the NP either does not significantly alter the energy of the emissive luminophore (excited or ground states), or Co is not proximate to the luminophore. We can further delineate these scenarios as 1) only a small population of Co exists on the NP surface (where the emitting state has been indicated to localize), 2) Co is oxidized on the surface of the particle and therefore does not interact with the luminophore of the NP, 3) Co is segregated into Co “islands” on the surface, 4) Co does not alter the energy of the emissive state in contrast to Cu in AuₓCu_{y}NPs and/or 5) Co does alter the energy of the emissive state, but at high % Co compositions, compositional heterogeneity and increasing oxidation confounds subsequent interpretation. Mechanism 4 is unlikely, given that all AuₓCo_{y}NPs exhibit an emission maximum that is blue-shifted from 100% AuNPs. HRTEM analysis indicates that the AuₓCo_{y}NPs do not exhibit large scale (i.e., observable) metal separation throughout the particle, which seems to eliminate mechanism 3. Based on our current experimental evidence, mechanisms 1 and 5 are the most probable explanations for the composition dependence of the maximum λ_{EM} from AuₓCo_{y}NPs.

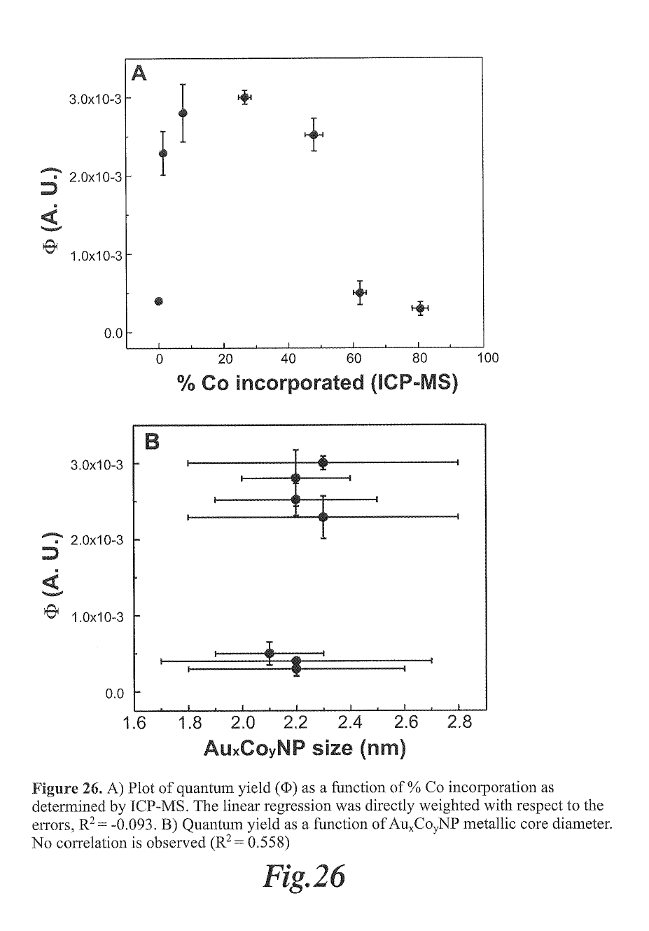

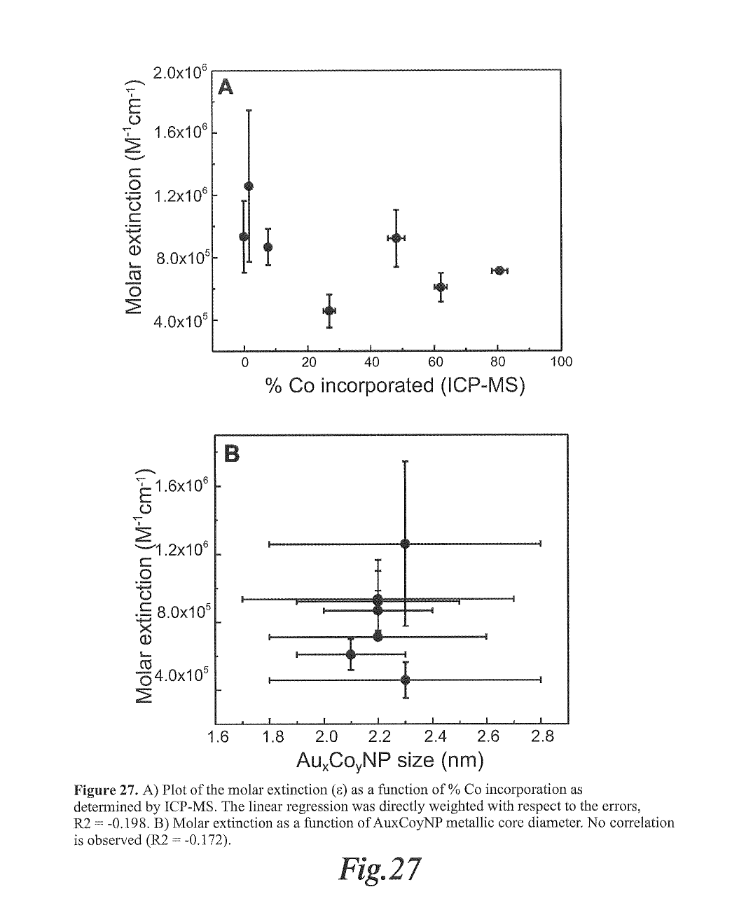

Although the definitive mechanism of PL for these small Au-transition metal NPs is still being determined, standard PL characterization is possible. Quantum yield (Φ) and molar extinction coefficient (ε) measurements were used to calculate particle brightness (ε×Φ). The brightness value determines the probability of absorbed and emitted of photons and is a useful figure of merit to compare luminophores. Measured quantum yield values are consistent with those found for other noble metal nanoparticle systems (Table 3). Quantum yield and brightness varied non-linearly (Table 3) as a function of composition (FIGS. 26A-28A) with the brightest particles containing ≈2% Co. AuₓCo_{y}NPs exhibit no observable size dependence of optical properties (FIG. 25, 26B-28B). Nanoparticle PL was evaluated in both D₂O and H₂O. D₂O was used to eliminate solvent absorption interference, however evaluation in H₂O was also conducted in order to facilitate comparison with other luminophores that have been measured in non-deuterated solvents. All optoelectronic properties were the same, within error, in both solvents. For comparison, an emission spectrum of AuₓCo_{y}NPs (y=48.1±2.7%) in H₂O is shown in FIG. 24. The AuₓCo_{y}NPs display brightness values that are over an order of magnitude higher than alternative biocompatible probes such as (Yb(III)TsoxMe), a sensitized lanthanide complex evaluated in water (2884 M⁻¹cm⁻¹ vs 83 M⁻¹ cm⁻¹).

The combination of magnetic and optical properties from AuₓCo_{y}NPs are clearly interesting for application as multimodal MRI contrast agents and therefore the relaxivity properties of each particle composition were also evaluated. Previous reports indicate that metallic Co T₂ relaxivities are both field strength and concentration dependent. To study the effect of field strength, the relaxivity of the AuₓCo_{y}NPs was measured at 37° C. at two different static fields, 0.47 T (20 MHz proton Larmor frequency) and 7 T (300 MHz proton Larmor frequency) (Table 7). As a control experiment, the relaxivity of 100% AuNPs was measured, and no effect on relaxivity was observed. For both field strengths, AuₓCo_{y}NPs had a significant effect on the transverse relaxation time (T₂) of water, and had little to no influence on the longitudinal relaxation time (T₁). These results indicate that AuₓCo_{y}NPs have the ability to maintain proton T₁ values that are the same as the surrounding tissue (providing essentially no positive contrast properties) while significantly dephasing the transverse magnetization used in MRI signal detection. This property most efficiently produces negative (dark) spots in the final image, making AuₓCo_{y}NPs attractive negative-T₂ contrast agents.

Even at low field strength, all AuₓCo_{y}NP compositions show very little effect on T₁, leading to r₂/r₁ values that, in all cases, are either comparable to or larger than those of a clinically available T₂ contrast agent, Ferumoxsil (SPION), which has a diameter nearly 3 times larger than the AuₓCo_{y}NP alloys reported here. The comparable or in some cases, enhanced, relaxivity for AuₓCo_{y}NPs (despite their smaller diameter compared to reported SPIONs) is likely the result of the higher saturation magnetization of Co compared to iron oxide (see below for a full comparison of AuₓCo_{y}NPs to previously reported iron oxide nanoparticles). Since tissues already have relatively short T₂ times (˜10²-10³ ms), in order to be considered an effective negative T₂ contrast agent, r₂ values must be orders of magnitude larger than r₁ values typically required for positive contrast agents. Further, as field strength is increased, T1 effects, as well as the efficiency of positive contrast agents, are expected to diminish. As clinical imaging instrumentation moves to higher field strengths to achieve greater resolution, the necessity to develop and implement improved contrast agents for T₂ weighted imaging becomes increasingly important.

AuₓCo_{y}NP alloys may provide a platform to achieve T₂ enhancements greater than those observed from SPIONs, while maintaining a small particle size for renal clearance. As expected, at 7 T longitudinal relaxation times in the presence of even the most concentrated AuₓCo_{y}NPs is equal to that of pure water (˜6 s at 7 T). Both the per-Co and per-particle T₂ relaxivities at 7 T are listed in Table 3. Relaxivity values are reported as per-particle relaxivity values, in addition to per-Co relaxivity values, to facilitate comparison between nanoparticles of different composition and size. The per-particle comparison is made here due to the difference between superparamagnetic nanoparticles and chelated-metal based contrast agents. For chelated-metal contrast agents, such as commercially available gadolinium-based agents, water protons bind to a single metal center, and therefore per-metal relaxivities are preferred. For superparamagnetic nanoparticles, the particle itself behaves as a large paramagnetic ion. Therefore, per-particle relaxivities provide a more accurate assessment of contrast agent efficiency in the case of nanoparticles (but with the caveat that larger particles will almost always exhibit higher relaxivities compared with smaller particles of the same material, and this relationship between particle size and per-particle relaxivity is not necessarily linear depending on the particle system). To compare AuₓCo_{y}NP T₂ relaxivities to other contrast agents, the per-particle relaxivity was calculated for reported earth-abundant metal nanoparticles of comparable size. Indeed, AuₓCo_{y}NPs exhibit comparable or enhanced per-particle T₂ relaxivities compared to SPIONs, despite the fact that AuₓCo_{y}NPs are smaller in diameter. Additionally, AuₓCo_{y}NPs show improved T₂ relaxivities compared to 0D and 1D gold-cobalt ferrite and gold-iron oxide heterostructures. Most relaxivities in the literature are reported as per-metal relaxivities. This figure of merit is important, as biological compatibility and toxicity is likely to be a function of transition metal concentration (for cobalt as well as iron), allowing a more straightforward assessment than particle concentration (although both cobalt and iron are used already in biomedical applications such as surgical implants).

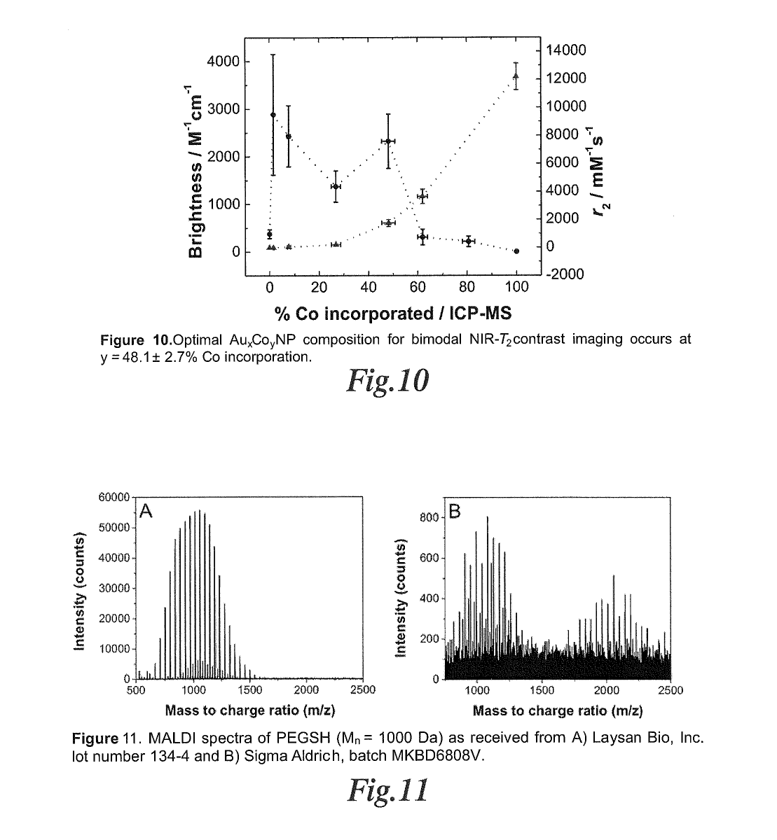

Because a wide range of AuₓCo_{y}NP compositions can be accessed via the current synthesis, the % Co incorporation parameter was explored to find a composition with both high NIR brightness and high T₂-relaxivity. This optimal composition can be determined by plotting r₂ at 7 T and NIR brightness as a function of % Co incorporation (FIG. 10). Particle brightness is highest for AuₓCo_{y}NPs (y=1.6±0.1%) and decreases until no NIR PL is observed. For per-particle relaxivity, as % Co incorporated increases, r₂ values become more favorable for negative MRI contrast. The trends for NIR brightness and r₂ intersect at approximately 55% Co incorporated in the particle. The particle composition closest to this value that retained desirable imaging properties was AuₓCo_{y}NPs, y=48.1±2.7%. Even at 48.1±2.7% Co incorporation, the per-particle relaxivity (r₂=1750 mM⁻¹s⁻¹) remains competitive compared to marketed negative contrast agents and exceeds the relaxivity values for reported iron oxide nanoparticles of similar sizes. Likewise, particle brightness (2322 M⁻¹cm⁻¹) also remains high when compared to other biocompatible NIR probes. For this reason, we conclude that 48.1±2.7% Co incorporation is an appropriate composition for a dual NIR-T₂ contrast imaging agent.

In summary, a preferred embodiment of the present disclosure a method for preparing a previously inaccessible library of composition tunable AuₓCo_{y}NP alloys is provided. This method can be used to tailor magnetic susceptibility while maintaining almost identical particle size and surface chemistry. It is believed that these particles have also enabled the first observation of photoluminescence from a Au—Co nanoparticle species at any size range or composition. Combined, these magnetic and optical features generate a promising multi-modal agent that exhibits NIR emission and MRI contrast properties that meet or exceed current standards, all at small particle diameters. Taken together, these data suggest that alloying behavior at the nanoscale may deviate significantly from bulk trends and that access to these new stoichiometries should yield an exciting diversity of unique, tunable physical properties useful in applications ranging from multimodal theranostics to heterogeneous catalysis.

### Experimental Section

Synthesis

Materials:

Hydrogen tetrachloroaurate(III) trihydrate (HAuCl₄ 3H₂O, 99.999%), cobalt(II) nitrate hexahydrate (Co(NO₃)₂ 6H₂O, >99.99%), sodium borohydride (NaBH₄, 99.99%), dimethyl sulfoxide (DMSO, >99.9%), were obtained from Sigma-Aldrich and used as received. Poly(ethylene glycol) methyl ether thiol (average Mₙ=1000 Da) was obtained from Laysan Bio, Inc. or Sigma Aldrich (see SI for discussion of heterogeneity in commercially available PEGSH) and used as received. Deuterium oxide (D₂O) and DMSO-d₆ were purchased from Cambridge Isotope Laboratories, Inc. and used as received. NANOpure (Thermo Scientific, >18.2 MΩ cm) water was used to prepare all solutions unless otherwise indicated. Before use, all glassware and Teflon coated stir bars were washed with aqua regia (3:1 ratio of concentrated HCl and HNO₃ by volume) and rinsed thoroughly with water. Caution: Aqua regia is highly toxic and corrosive and requires proper personal protective equipment. Aqua regia should be handled in a fume hood only.

Synthesis of AuₓCo_{y}NPs:

AuₓCo_{y}NP alloys were synthesized by co-reduction of HAuCl₄ and Co(NO₃)₂ with NaBH₄ at room temperature in an aqueous solution containing the capping ligand, poly(ethylene glycol) methyl ether thiol (PEGSH, average Mₙ=1000 Da). Reagents were added to a glass vial, while stirring, in the following order: water (4.29 mL), HAuCl₄ (X mL, 20.0 mM), Co(NO₃)₂ (X mL, 20.0 mM), PEGSH (375 μL of 10.0 mM), and NaBH₄ (450 μL of 20.0 mM) (N. B. volume of metal stock added for each composition is listed in Table 4). The total concentration of metal cations was held constant while the molar ratio of Au and Co was varied. The initial molar ratio of Co to Au was varied from 0-100%, while maintaining the same total metal, capping ligand, and reducing agent concentrations.

Nanoparticle Purification:

The entire contents of the NP synthesis were transferred to Amicon Ultra-4 Ultracel 10 kDa molecular weight cutoff centrifugal filters (Merck Millipore Ltd.). Samples were purified from excess PEGSH and metal salts using an Eppendorf 5804 or 5804R centrifuge with swing bucket rotor (A-44-4) (Eppendorf, Inc.) with a force of 4000 RFC at 20° C. for 12-15 min. The resulting concentrated particles (typically ≈50 μL in water) were diluted in the concentrator tube to a volume of 3 mL with water. The loose pellet was resuspended by gentle mixing using a pipette prior to re-centrifugation. This washing procedure was repeated 5 times. Purified AuₓCo_{y}NPs were then characterized by electron microscopy techniques, UV-Visible spectroscopy, ICP-MS, XPS, photoluminescence, and ¹H NMR techniques.

Characterization

Electron Microscopy:

Samples were prepared for electron microscopy by drop casting an aliquot of purified NP solution (diluted 1:10 or 1:100 with water) onto ultra-thin (3-5 nm) carbon type A 400 mesh copper grids (Ted Pella, Inc.). Samples were allowed to slowly air dry for at least 10 hours followed by drying under vacuum. Bright field, HRTEM and STEM characterization was performed using a JEOL JEM-2100F equipped with a Gatan GIF-Tridiem camera and Oxford Inca EDS detector operating at 200 kV (NanoScale Fabrication and Characterization Facility, Petersen Institute of NanoScience and Engineering, Pittsburgh, Pa.).

Size Determination by NMR:

Pulsed field gradient stimulated echo (PFGSE) ¹H NMR measurements were performed on a Bruker 500 Ultrashield magnet with an AVANCE III 500 Console or a Bruker 600 Ultrashield magnet with an AVANCE III 600 Console (Bruker Biospin, Billerica, Mass.) at 298 K. AuₓCo_{y}NPs NMR samples were lyophilized, resuspended in DMSO-d₆, and loaded in a 5 mm NMR tube for measurement. ¹H NMR diffusion spectra were acquired on a broadband observe probe using a stimulated echo bipolar pulsed field gradient pulse sequence (for calculations and additional analysis, see below).

XPS Analysis:

XPS was performed using a Thermo Scientific K-Alpha with monochromatic Al Kα X-rays (RJ Lee Group, Inc., Monroeville, Pa.). Survey and high resolution spectra were collected with a pass energy of 200 eV and 50 eV, respectively. Lyophilized NPs were resuspended in absolute ethanol and drop cast onto silicon wafers (University Wafer, Boston, Mass.). Prior to XPS collection, samples were sputtered for 30 seconds with an argon ion gun. All XPS spectra were measured with a 400 μm X-ray spot size. High resolution XPS spectra were charge referenced to the adventitious hydrocarbon C1s peak at 284.8 eV.

ICP-MS Analysis:

ICP-MS analysis was performed using an Argon flow with a Nexion spectrometer (PerkinElmer, Inc.). An ultrapure aqua regia solution was prepared with a 3:1 ratio of hydrochloric acid (Sigma Aldrich >99.999% trace metal basis): nitric acid (Sigma Aldrich, >99.999% trace metal basis), a portion of which was diluted with NANOpure water for a 5% v/v aqua regia matrix. An aliquot of the purified nanoparticle samples was digested with ≈100 μL of ultrapure, concentrated aqua regia in a 10 mL volumetric flask, and diluted to volume with the 5% aqua regia solution. The unknown Au and Co concentrations were determined by comparison to a 5-point standard calibration curve with a range of 1-30 ppb prepared from a gold standard for ICP (Fluka, TraceCERT 1001±2 mg/L Au in HCl) and a cobalt standard for ICP (Fluka, TraceCERT 1000±2 mg/L Co in HNO₃), respectively, and diluted in the 5% aqua regia matrix. The ICP standards were measured 5 times and averaged, while all unknown samples were measured in triplicate and averaged. An 8 minute flush time with 5% aqua regia matrix was used between all runs, and a blank was run before every unknown sample to confirm removal of all residual metals.

Magnetic Susceptibility Measurements:

Mass magnetic susceptibility for NPs were recorded on a Bruker 600 Ultrashield magnet (14.1 T) with an AVANCE III 600 Console or a Bruker 700 Ultrashield magnet (16.4 T) with an AVANCE III 700 Console (Bruker Biospin, Billerica, Mass.) equipped with a BVT3000 and BCU05 variable temperature unit, respectively. ¹H NMR spectra were collected at 298 K using the Evans' method.^{[25]} AuₓCo_{y}NPs were synthesized and purified and the concentrated NP pellet was lyophilized. The mass of the dried NPs was recorded and resuspended in ¹mL of D₂O and loaded into a 5 mm NMR tube along with an internal sealed capillary tube of pure D₂O. A 1D ¹H NMR spectrum of each sample was recorded with 16 transients. ¹H NMR chemical shifts were referenced to the HDO peak from the capillary at 4.7 ppm. Typical 90° radiofrequency pulses were −9 μs for ¹H NMR spectra, and were processed using Bruker Topspin 3.0 and iNMR. The distance in Hz between the residual HDO peak of the pure D₂O and the HDO peak of the D₂O containing the AuₓCo_{y} colloidal suspension (experimental ¹H NMR spectra shown in Figure S9) was measured and used to calculate the magnetic susceptibility (see SI for additional calculation details).

UV-Visible Spectroscopy:

Molar Extinction Coefficient: Nanoparticle extinction coefficients were calculated using the UV-vis-NIR spectrum of the NPs after purification. Spectra were taken using a Cary 5000 UV-vis-NIR (Agilent, Inc.). UV-vis measurements were collected of nanoparticle suspensions diluted in D₂O using 1.0 cm quartz cuvettes (Hellma, Inc.).

Photoluminescence: Quantum Yield and Brightness:

NP suspensions in D₂O were prepared from the purified AuₓCo_{y}NP stocks at concentrations ≦0.25 abs at 340 nm determined by UV-Vis. Emission spectra were acquired on a HORIBA Jobin Yvon IBH FluoroLog-322 spectrofluorometer equipped with a Hamamatsu R928 detector for the visible domain; DSS-IGA020L (Electro-Optical Systems, Inc.) detector for the NIR domain and a temperature controller using 1.0 cm×0.4 cm quartz cuvettes (Hellma, Inc). A 780 nm NIR cut-on filter (Newport FSQ-RG780, Newport Corporation, Inc.) was used to block the excitation source. The quantum yields in the NIR region were determined by the optically dilute method. Excitation spectra of the purified AuₓCo_{y}NPs were collected using an emission slit of 20 nm centered at 950 nm with an excitation slit of 5 nm. Spectra were collected in 1 nm increments using an integration time of 0.4 s from 290-600 nm and the NIR cut-on (780 nm) filter was used to filter the emission (Figure S19). Excitation spectra have been corrected for lamp power fluctuations and the instrument response.

Relaxivity Measurements:

Longitudinal (T₁) and transverse (T₂) relaxation time measurements were collected for five dilutions of each sample at 37° C. using an inversion recovery pulse sequence and the Carr-Purcell-Meiboom-Gill (CPMG) spin echo pulse sequence, respectively. Relaxation measurements were collected at both 20 MHz (0.47 T) on a Bruker mq20 minispec NMR analyzer and 300 MHz (7 T) on a Bruker DRX 300 MHz magnet. In order to minimize radiation damping effects at 7 T, the NPs were suspended in 50/50 H₂O/D₂O and the probe was de-tuned prior to measurement. All relaxivity measurements were performed in triplicate (three independent syntheses of each composition), with ICP-MS analysis of each sample for exact metal concentration.

Synthesis of Gold-Cobalt Nanoparticle Alloys.

Nanoparticles were synthesized in water at room temperature in air. Reagents were added to a glass vial, while stirring, in the following order: 4.29 mL water, 20.0 mM HAuCl₄, 20.0 mM Co(NO₃)₂, 375 μL of 10.0 mM PEGSH, and 450 μL of 20.0 mM NaBH₄ (volume of metal stock added is listed in Table 4) The total concentration of metal cations was held constant while the molar ratio of gold and cobalt was varied (Table 4. Initial reaction conditions for AuₓCo_{y}NP synthesis).

TABLE 4
Initial reaction conditions for AuₓCo_{y}NP synthesis.

| Co (%) | 20.0 mM Au³⁺ (μL) | 20.0 mM Co²⁺ (μL) |
|---|---|---|
| 0 | 188.0 | 0 |
| 50 | 94.0 | 94.0 |
| 60 | 75.2 | 112.8 |
| 70 | 56.4 | 131.6 |
| 80 | 37.6 | 150.4 |
| 85 | 28.2 | 159.8 |
| 90 | 18.8 | 169.2 |
| 100 | 0 | 188 |

After thoroughly mixing the gold and cobalt solution, 375 μL of 10 mM PEGSH solution was quickly added to the solution while stirring. After PEGSH addition, the solution was reduced by rapid injection of 450 μL of a 20.0 mM solution of fresh, ice cold NaBH4 while vigorously stirring for 1 minute. The capped vials were “aged” on the benchtop for at least 1 hour prior to purification.

Note on PEGSH Supplier and Impact on Particle Properties:

The MALDI spectra of PEGSH from two vendors were recorded in an α-cyano-4-hydroxycinnamic acid (CHCA) matrix on a 100 well plate with an accelerating voltage of 20,000 V. We noticed that optical properties varied based on the supplier of the PEGSH used in synthesis. Magnetic properties, crystallographic features, and particle size did not vary within error. FIG. 11 shows the MALDI spectra of PEGSH (Mₙ=1000 Da) obtained from Laysan Bio, Inc. (11A) and Sigma-Aldrich (11B). From these spectra, it is clear that the Sigma-Aldrich PEGSH contains a substantially higher percentage of dimerized compounds with a broader molecular weight distribution when compared to material obtained from Laysan Bio, Inc. The impact of these variations on PL, but not on particle size and crystal structure is in agreement with the surface-thiol based PL mechanisms proposed.

Nanoparticle Purification.

The entire contents of the NP synthesis were transferred to Amicon Ultra-4 Ultracel 10 kDa molecular weight cutoff centrifugal filters (Merck Millipore Ltd.). Samples were purified from excess PEGSH and metal salts using an Eppendorf 5804 or 58048 centrifuge with swing bucket rotor (A-44-4) (Eppendorf, Inc.) with a force of 4000 RFC at 20° C. for 12-15 min. The resulting concentrated particles (typically ˜50 μL in water) were diluted in the concentrator tube to a volume of 3 mL with NANOpure water. The loose pellet was resuspended by gentle mixing using a pipette prior to re-centrifugation. This washing procedure was repeated 5 times. Purified AuₓCo_{y}NPs were then characterized by electron microscopy techniques, UV-visible spectroscopy, ICP-MS, XPS, PL, and ¹H NMR techniques.

TEM.

An aliquot from the purified NP solution was diluted 1:10 or 1:100 with NANOpure water prior to drop casting onto ultra-thin (3-5 nm) carbon type A 400 mesh copper grids (Ted Pella, Inc.). Samples were allowed to slowly air dry for at least 10 hours followed by drying under vacuum. High resolution transmission electron microscopy (HRTEM) characterization 5 was performed on a JEOL JEM-2100F equipped with a Gatan GIF-Tridiem camera and Oxford Inca EDS detector (NanoScale Fabrication and Characterization Facility, Petersen Institute of NanoScience and Engineering, Pittsburgh, Pa.). All instruments were operated at 200 kV. Scanning transmission electron microscopy characterization was performed using the JEM-2100F.

HRTEM: Analysis of Lattice.

The lattice fringes observed in the HRTEM were converted into reciprocal space using a fast Fourier transform function in the Digital Micrograph v2.30.542.0 (Gatan, Inc.) and/or ImageJ v 1.47d (National Institutes of Health). The angles between the spots and distance from the center of the reciprocal lattice were measured to determine the visible lattice planes and the zone axis using standard face-centered cubic (FCC) diffraction patterns to index the spots. The overall lattice constant for the particle was then determined for multiple (N>6) particles and reported as the average and standard error. Examples of the measurements for each evaluated AuₓCo_{y}NP alloy composition are shown in FIG. 12.

HRTEM: Size Distribution.

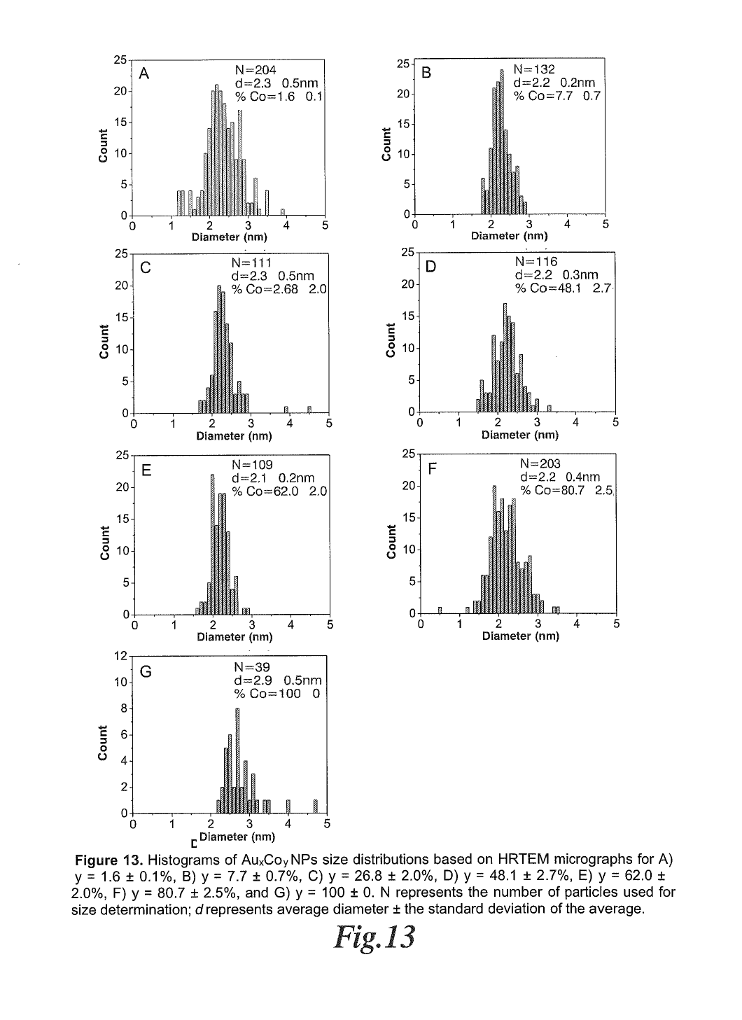

The metallic core size distribution of the NPs was determined by measuring the diameter of >100 NPs from various areas of the grid. ImageJ 1.47d (National Institutes of Health) was used to measure and count all particles. Average diameter and standard deviation were determined from the histogram plots (FIG. 13). Contrast, and therefore particle counts, were low for 100% CoNPs, HRTEM micrographs were still used for sizing, and these sizes were supported by measurements of particle hydrodynamic radii using PFGSE NMR (vide infra).

PFGSE ¹H NMR: Hydrodynamic Diameter Evaluation.

Pulsed field gradient stimulated echo (PFGSE) ¹H NMR measurements were performed on a Bruker 500 Ultrashield™ magnet with an AVANCE III 500 Console or a Bruker 600 Ultrashield™ magnet with an AVANCE III 600 Console (Bruker Biospin, Billerica, Mass.) at 298 K. AuₓCo_{y}NPs NMR samples were lyophilized, resuspended in DMSO-d₆, and loaded in a 5 mm NMR tube for measurement. ¹H NMR diffusion spectra were acquired on a broadband observe probe using a stimulated echo bipolar pulsed field gradient pulse sequence.

For a diffusing species examined with a stimulated echo bipolar pulsed field gradient sequence, the NMR signal intensity (I) is given by a modified Stejskal-Tanner equation.

$$I = {I}_{0} exp ( - {( γ G δ )}^{2} ( Δ - \frac{τ}{2} - \frac{δ}{8} ) D ) \qquad ( 1 )$$

Where I₀ is the initial intensity, γ is the gyromagnetic ratio of ¹H, G is the applied gradient strength, δ is the length of the gradient pulse, Δ is the diffusion time, τ is the time between bipolar gradient pulses, and D is the apparent diffusion coefficient. Rearranging this equation gives

$$ln ( \frac{I}{{I}_{0}} ) = - {( γ G δ )}^{2} ( Δ - \frac{τ}{2} - \frac{δ}{8} ) D ) = - kD \qquad ( 2 )$$

A plot of ln(I/I₀) vs k allows for direct evaluation of the diffusion coefficient. Using the Stokes-Einstein equation allows the assignment of a hydrodynamic radius (r_{H})

$${r}_{H} = \frac{{k}_{B} T}{6 π η D} \qquad ( 3 )$$

where k_{B} is the Boltzmann constant, T is the temperature in Kelvin, and η is the viscosity of the solvent. In order to accurately size the particles, the residual protonated solvent, DMSO, was used as a reference molecule to calculate the particle radius (r_{particle}) with the following relation:

$${r}_{particle} = \frac{{D}_{DMSO}}{{D}_{particle}} \cdot {r}_{DMSO} \qquad ( 4 )$$

A reference molecule for the hydrodynamic radius was used to eliminate discrepancies in the apparent diffusion coefficient due to variation in solution viscosity between samples, and temperature gradients across the sample during the course of the experiment. DMSO was chosen as the reference molecule because of its known hydrodynamic radius and distinct ¹H resonance from the capping ligand.

To address the issue of polydispersity in the nanoparticle sample, the signal intensity of the ¹H resonances were treated as a distribution of diffusion coefficients given by the following equation:

$$\frac{I}{{I}_{0}} = {\int }_{0}^{∞} P ( D ) exp ( - kD ) d D \qquad ( 5 )$$

This integral is the autocorrelation function for a system described by equation 2. However, the integral represents an ill-posed problem and cannot be evaluated reliably. A mathematical algorithm was developed and executed in MatLab R2010a, which requires an initial value for the diffusion coefficient and standard deviation. Since NMR signal intensity reflects the population distribution of an ensemble, equation 2 provides the average diffusion coefficient for the sample, allowing explicit control of the standard deviation and probability distribution to fit the autocorrelation function from raw NMR data. The standard deviation estimate can be manipulated to best fit any distribution. In this case, a Gaussian distribution was assumed. This provides the ability to adjust the distribution to determine the most appropriate model for each system and extract a reliable standard deviation.

STEM-EDS: Point STEM-EDS Analysis.

Representative STEM-HAADF and -EDS images from the JEOL JEM-2100F operated at 200 kV are shown in FIG. 14. A spot size of 1.5 nm and X tilt of 14° toward the X-ray Oxford INCA detector was used to generate EDS spectra. The STEM-EDS values are an average of at least four measurements (Table 5) for a single sample preparation unless indicated otherwise. The associated error with EDS molar ratio is reported as the standard deviation of the measurement. The error for ICP-MS represents the standard deviation in the ICP-MS measurement. The variation of the % Co is inhomogeneous which may indicate a variety of compositions within a single sample. However, the EDS signal is also weak which likely contributes to the large variation in the % Co from particle to particle. The variation in the relative composition of small (1-3 nm diameter) bimetallic alloys and that of STEM-EDS from bulk compositional analysis have been observed before.^{[2]} Ultimately, however, comparison between ICP-MS analysis of the bulk colloid and point STEM-EDS averaged over several different particles (that exhibited quantifiable Co K and Au L characteristic X-ray edges), indicates that the % Co incorporation measured with the two methods are similar.

TABLE 5
Comparison of AuₓCo_{y}NP composition
measured by ICP-MS and STEM-EDS.

| NP composition (% Co) | NP composition (% Co) |
|---|---|
| ICP-MS | STEM-EDS |
| 25.7 ± 1.0 | 22.5 ± 12.3 |
| 37.1 ± 0.4 | 36.4 ± 13.5 |

Note on Powder X-Ray Diffraction at Small, Discrete Particle Sizes (d<3 nm).

We note in the text that comparison of XRD spectra from particles of this size range was not possible due to significant line broadening. This result is consistent with mathematical predictions for particles with diameters less than 3 nm. For example, the Scherrer equation predicts that the FWHM of the diffraction line more than doubles simply by decreasing particle diameter from 5 nm to 2.2 nm. This line broadening at small crystallite size is further exacerbated by lattice strain and deviations in d-spacing as a function of curvature and alloying, which together can cause an additive, linear increase in FWHM with increased strain. Our particles are both small (d=2.1-2.3 nm) and are known to exhibit lattice strain even at 100% Au (100% AuNPs, lattice constant=3.96 Å, compared to bulk Au_{fcc}=4.079 Å). Therefore, while broadening and diffraction pattern shifts are highly useful metrics to assess alloy formation and phase in many cases, for our system, it proved limited.

X-Ray Photoelectron Spectroscopy.

X-ray photoelectron spectroscopy (XPS) was performed using a Thermo Scientific K-Alpha with monochromatic Al Kα X-rays (RJ Lee Group, Inc., Monroeville, Pa.). Survey and high resolution spectra were collected with a pass energy of 200 eV and 50 eV, respectively. Lyophilized NPs were resuspended in absolute ethanol and drop cast onto cleaned (for ultra-high vacuum) silicon (p-doped (boron)) wafers (University Wafer, Boston, Mass.). Prior to XPS collection, samples were sputtered for 30 seconds with an argon ion gun. All XPS spectra were measured with a 400 μm X-ray spot size. High resolution XPS spectra were charge referenced to the adventitious hydrocarbon 1s peak at 284.8 eV. The survey XPS spectrum for AuₓCo_{y}NPs (y=48.1±2.7%) is shown in FIG. 15. All NP compositions showed Au, Co, C, O, and S, in the XPS survey spectra, with the exception of AuNPs and CoNPs, which lacked Co and Au, respectively.

High resolution XPS spectra were also collected for Au4f, Co2p, O1s, C1s, and S2p to analyze metal oxidation state, as well as any alloying effect on binding energy. FIG. 16 shows high resolution XPS spectra for Au4f and Co2p for all NP compositions. In both cases, metallic Au and metallic Co are observed. A shift to higher binding energy is observed for Au4f peaks as the % Co increases. On the other hand, Co2p peaks shift to lower energy as the extent of Au incorporation increases from 0% in pure CoNPs. These observations are consistent with, but not definitive for, alloy formation.

A similar synthetic method of metal reduction in aqueous solution with NaBH4 has been reported to form metal-boride species, in particular cobalt borides.^{[5]} To confirm that no metalboride species were formed during our synthesis, additional high resolution spectra were recorded in the boron chemical shift range. FIG. 17 shows high resolution B1s spectra of H₃BO₃, AuNPs, CoNPs, and AuₓCo_{y}NPs (y=48.1±2.7%). CoxB species show chemical shifts from ˜188-189 eV.^{[6]} No peaks are observed in any of the nanoparticle spectra, ruling out the formation of metal-borides at a measurable concentration.

ICP-MS Analysis.

ICP-MS analysis was performed using an argon flow with a Nexion spectrometer (PerkinElmer, Inc.). An ultrapure aqua regia solution was prepared with a 3:1 ratio of hydrochloric acid (Sigma-Aldrich >99.999% trace metal basis): nitric acid (Sigma Aldrich, >99.999% trace metal basis), a portion of which was diluted with NANOpure water for a 5% v/v aqua regia matrix. A small aliquot of the purified nanoparticle samples was digested with ˜100 μL of ultrapure, concentrated aqua regia in a 10 mL volumetric flask and diluted to volume with the 5% aqua regia solution. The unknown Au and Co concentrations were determined by comparison to a 5-point standard calibration curve with a range of 1-30 ppb prepared from a gold standard for ICP (Fluka, TraceCERT 1001±2 mg/L Au in HCl) and a cobalt standard for ICP (Fluka, TraceCERT 1000±2 mg/L Co in HNO₃), respectively, and diluted in the 5% aqua regia matrix. The ICP standards were measured 5 times and averaged, while all unknown samples were measured in triplicate and averaged. An 8 minute flush time with 5% aqua regia matrix was used between all runs, and a blank was run before every unknown sample to confirm removal of all residual metals. Averaged values and standard deviations from at least five independent syntheses are shown in Table 6. The % Co incorporated (measured with ICP-MS) vs. the initial % Co added during synthesis is plotted in FIG. 18.

TABLE 6
Initial molar % Co added during synthesis and
final % Co incorporation in the NP by ICP-MS analysis.
Error represents the standard error.

| Co added during synthesis (%) | Co incorporated in final NP (%) |
|---|---|
| 0 | 0 ± 0 |
| 50 | 1.6 ± 0.1 |
| 60 | 7.7 ± 0.7 |
| 70 | 26.8 ± 1.9 |
| 80 | 48.1 ± 2.7 |
| 85 | 62.0 ± 2.0 |
| 90 | 80.7 ± 2.5 |
| 95 | 89.8 ± 1.2 |
| 100 | 100 ± 0 |

As mentioned in the main text, little to no cobalt incorporation was observed until an initial molar ratio of 50% Co. This initial lag in Co incorporation may be a product of the disparity in reduction potential between Co²+/Co⁰ (E⁰=−0.28 V vs. NHE) and Au³+/Au⁰ (E⁰=+1.50 V vs. NHE), which results in less available Co monomer (here, referring to “monomer” as described by La Mer). Previous reports indicate that co-reduction during nucleation was a crucial factor in the formation of intermetallics. Differences in reduction potential are also thought to play a large role in the formation of core-shell particles or incomplete mixing of the two components (e.g. heterogeneous solid solution or “island” formation). We hypothesize that above 50% Co initial molar ratios, a threshold amount of Co monomer is available to co-nucleate along with Au monomer, allowing both elements to be incorporated into a single particle.

Magnetic Susceptibility Measurements.

Mass magnetic susceptibility for NPs were recorded on a Bruker 600 Ultrashield™ magnet (14.1 T) with AVANCE III 600 Console or a Bruker 700 Ultrashield™ magnet (16.4 T) with AVANCE III 700 Console (Bruker Biospin, Billerica, Mass.) equipped with a BVT3000 and BCU05 variable temperature unit, respectively. ¹H NMR spectra were collected at 298 K using the Evans' method.^{[10]} AuxCoyNPs were synthesized and purified and the concentrated NP pellet was lyophilized. The mass of the dried NPs was recorded and resuspended in 1 mL of D₂O and loaded into a 5 mm NMR tube along with an internal sealed capillary tube of pure D₂O. A 1D ¹H NMR spectrum of each sample was recorded with transients. ¹H NMR chemical shifts were referenced to the HDO peak from the capillary at 4.7 ppm. Typical 90° radiofrequency pulses were ˜9 μs for ¹H. NMR spectra were processed using Bruker Topspin 3.0 and iNMR. The distance in Hz between the residual HDO peak of the pure D₂O and the HDO peak of the D₂O containing the AuₓCo_{y} colloidal suspension (experimental ¹H NMR spectra shown in FIG. 19) was measured and used to calculate the magnetic susceptibility according to the following equation (6):

$${X}_{tot , g} = \frac{3 Δ f}{4 π fm} + {X}_{0} \qquad ( 6 )$$

Equation 6 is a modified Evans' method equation to calculate the mass susceptibility using modern NMR spectrometers and cylindrical NMR tubes where Δf is the distance in Hz between the residual HDO peak of the pure D₂O and the HDO peak of the D₂O containing the material of interest, f is the operating frequency of the NMR spectrometer in Hz, m is the mass of material that is suspended in 1 mL of solvent, and X₀ is the mass susceptibility of the solvent (D₂O=−6.466×10⁻⁷ cm³/g). This method measures the total susceptibility of the colloid, which is the sum of the paramagnetic and diamagnetic contributions. The magnetic susceptibility of numerous AuₓCo_{y}NP compositions is shown in FIG. 20.

¹H NMR: Molecular Characterization.

All AuₓCo_{y}NPs were characterized with ¹H NMR to assess the integrity of the ligand shell as well as the purity of the colloidal solution (FIG. 21). From ¹H NMR analyses, we confirmed that all of the PEGSH (within the detection limit of NMR) was particle-bound, as evidenced by the significant dephasing of ¹H resonances of the CH₂ protons adjacent to the thiol. In FIG. 21, the triplet at 2.67 ppm (CH₂ protons adjacent to thiol) are not present in the particle-bound PEGSH spectra, as indicated by the dotted line. The CH₂ protons adjacent to the thiol are not observed as a result of heterogeneous line-broadening from chemical shift distribution of various chemisorption sites, and proximity to a paramagnetic center (in the case of particles displaying positive magnetic susceptibility values). The spectral window was expanded to 250 ppm during acquisition to search for hyperfine-shifted peaks present from the formation of high-spin Co²⁺ complex impurities. No ¹H NMR spectral changes in chemical shift were observed. The spectra are consistent with our finding of T₂-enhancing AuₓCo_{y}NPs. As % Co increases, we observe increased line-broadening of the ligand ¹H NMR peaks.

UV-Visible Spectroscopy: Molar Extinction Coefficient.

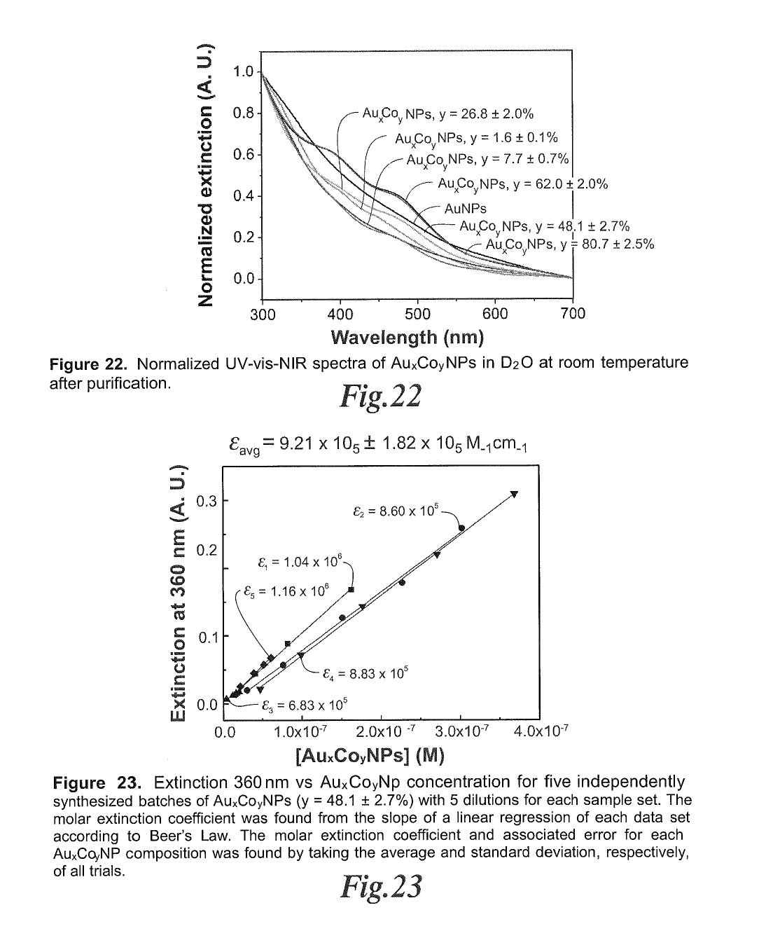

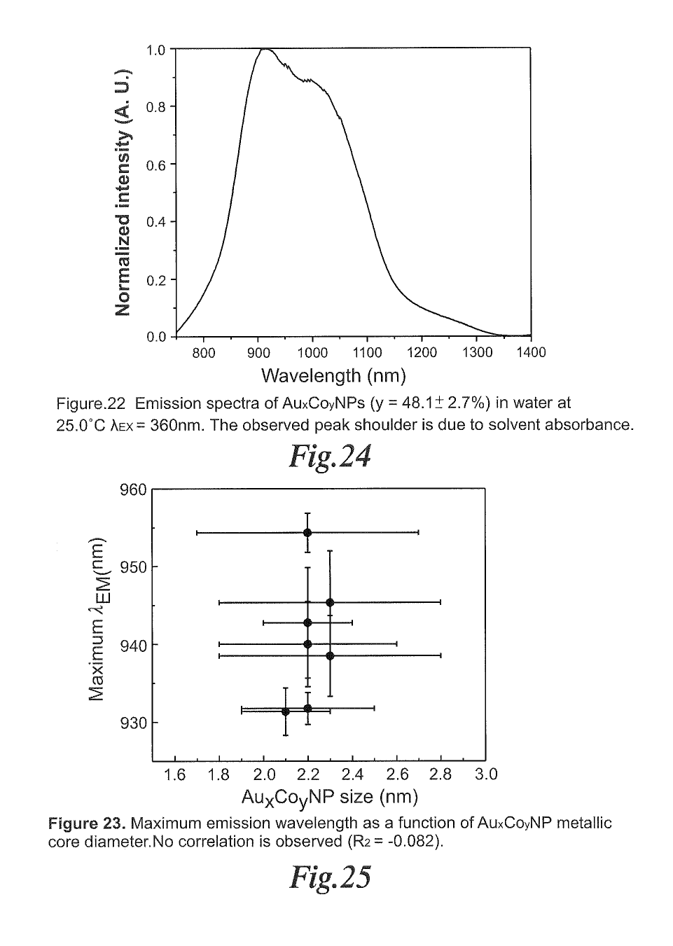

Nanoparticle extinction coefficients were calculated using the UV-vis-NIR spectrum of the NPs after purification. Spectra were taken using a Cary 5000 UV-vis-NIR (Agilent, Inc.). UV-vis measurements were collected of nanoparticle suspensions diluted in D₂O using 1.0 cm quartz cuvettes (Hellma, Inc.) The UV22 visible spectra of the AuₓCo_{y}NPs exhibit a relatively featureless extinction at low % Co incorporations, with gradual emergence of peaks at ˜390 nm and ˜470 nm as shown in FIG. 22. The peaks were not observed in controls of Co(NO₃)₂, or Co(NO₃)₂+PEGSH, indicating that they are characteristic of a Co species contained in the particle.

To calculate molar extinction coefficient, particle concentration was calculated from the concentration of the metals as determined by ICP-MS and the average diameter of the NPs determined using HRTEM micrographs. A more detailed flow chart describing this analysis is presented elsewhere. The total number of metal atoms was estimated per particle by dividing the volume of the NP sphere by the volume of the FCC lattice calculated for each composition. Aliquots of concentrated, purified nanoparticle solutions were diluted to 5 different concentrations and the UV-vis-NIR spectrum recorded. From a plot of extinction at 360 nm vs. particle concentration, the molar extinction coefficient was extracted using Beer's Law (A=εbc). An example plot is shown in FIG. 23 for AuₓCo_{y}NPs (y=48.1±2.7%). The reported molar extinction coefficient is the average of three or more independently synthesized batches of AuₓCo_{y}NPs and the estimated error in ε was determined from the standard deviation.

Photoluminescence: Quantum Yield and Brightness.

NP suspensions in D₂O were prepared from the purified AuₓCo_{y}NP stocks at concentrations ≦0.25 abs at 340 nm determined by UV-vis. Emission spectra were acquired on a HORIBA Jobin Yvon IBH FluoroLog3-221 spectrofluorometer equipped with a Hamamatsu R928 detector for the visible domain; DSS-IGA020L (Electro-Optical Systems, Inc.) detector for the NIR domain and a temperature controller using 1.0 cm×0.4 cm quartz cuvettes (Hellma, Inc). A 780 nm NIR cut-on (Newport FSQ-RG780, Newport Corporation, Inc.) filter was used to block the excitation source. The quantum yields in the NIR region were determined by the optically dilute method using the following equation (7):

$$\frac{Φ}{{Φ}_{r}} = [ \frac{{A}_{r} ( {λ}_{r} )}{{A}_{x} ( {λ}_{x} )} ] [ \frac{I ( {λ}_{r} )}{I ( {λ}_{x} )} ] [ \frac{n \frac{2}{x}}{n \frac{2}{r}} ] [ \frac{{D}_{x}}{{D}_{r}} ] \qquad ( 7 )$$

where A is the absorbance at the excitation wavelength (λ), I is the intensity of the excitation light at the same wavelength, n is the refractive index, and D is the integrated luminescence intensity (780-1450 nm). The subscripts ‘x’ and ‘r’ refer to the sample and reference, respectively. The reference used in this study was [Yb(tropolone)₄]⁻ in dry DMSO as a standard (Φᵣ=0.019) following synthesis as outlined in the literature.^{[16]} Refractive index values of n=1.333 (AuNP in D₂O) and n=1.479 (DMSO) were used. The estimated error in Φₓ was determined by the standard deviation relative to the average for measurements of three independently synthesized batches of AuₓCo_{y}NPs using corrected emission spectra. The brightness was determined from the molar extinction coefficient and quantum yield (brightness=ε×Φ) and the error propagated from the standard deviations. Emission spectra for AuₓCo_{y}NPs are shown in FIG. 3. All emission spectra were recorded in D₂O to minimize solvent interference. Since the application of AuₓCo_{y}NPs as dual imaging agents will require acquisition in H₂O rather than D₂O, we also recorded the emission spectrum of AuₓCo_{y}NP, y=48.1±2.7% in water. The emission spectra of AuₓCo_{y}NPs (y=48.1±2.7%) in water is shown in FIG. 24.

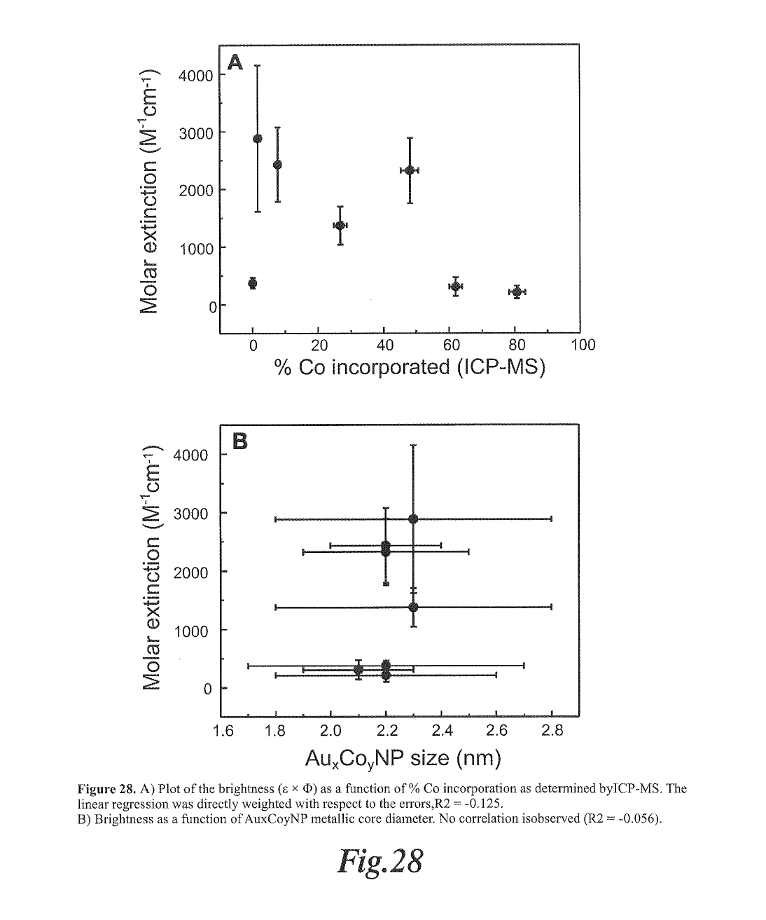

The emission maxima (λ_{EM}) of the spectra were calculated by visually determining the wavelength at which the large emission intensity occurred. At least three of these emission maxima measurements were made for each composition. The standard error of the mean (λ_{EM}) is reported in Table 3. No correlation was evident between maximum λ_{EM} and nanoparticle core diameter (FIG. 25). For each of PL figure of merit (Φ, ε and brightness) we compare these values individually with both % Co and average particle size, to determine possible correlations between these variables (FIGS. 26-28). The brightness and molar extinction of the AuₓCo_{y}NPs showed a non-linear dependence on % Co with a peak brightness and ε at 1-3% Co (FIG. 28). No dependence of optoelectronic properties and particle size were observed.

Excitation Spectra.

Excitation spectra of the purified AuₓCo_{y}NPs were collected using an emission slit of 20 nm centered at 950 nm with an excitation slit of 5 nm. Spectra were collected in 1 nm increments using an integration time of 0.4 s from 290-600 nm and the NIR cut-on (780 nm) filter was used to filter the emission (FIG. 29). Excitation spectra have been corrected for lamp power fluctuations and the instrument response.

Relaxivity Measurements.

Longitudinal (T₁) and transverse (T₂) relaxation time measurements were collected for five dilutions of each sample at 37° C. using an inversion recovery pulse sequence and the Carr-Purcell-Meiboom-Gill (CPMG) spin echo pulse sequence, respectively. Relaxation measurements were collected at both 20 MHz (0.47 T) on a Bruker mq20 minispec NMR analyzer and 300 MHz (7 T) on a Bruker DRX 300 MHz magnet. In order to minimize radiation damping effects at 7 T, the NPs were suspended in 50/50 H₂O/D₂O and the probe was de-tuned prior to measurement. All relaxivity measurements were performed in triplicate (three independent syntheses of each composition), with ICP-MS analysis of each sample for exact metal concentration. Inverse relaxation times (1/T_{n=1,2}) vs. [AuₓCo_{y}NPs] or [Co] were plotted and fit to equation 8 to determine relaxivity (r₁ and r₂):

$$\frac{1}{{T}_{n} = 1 , 2} = \frac{1}{{T}_{n = 1 , 2}^{solvent}} + {r}_{n = 1 , 2} [ agent ] \qquad ( 8 )$$

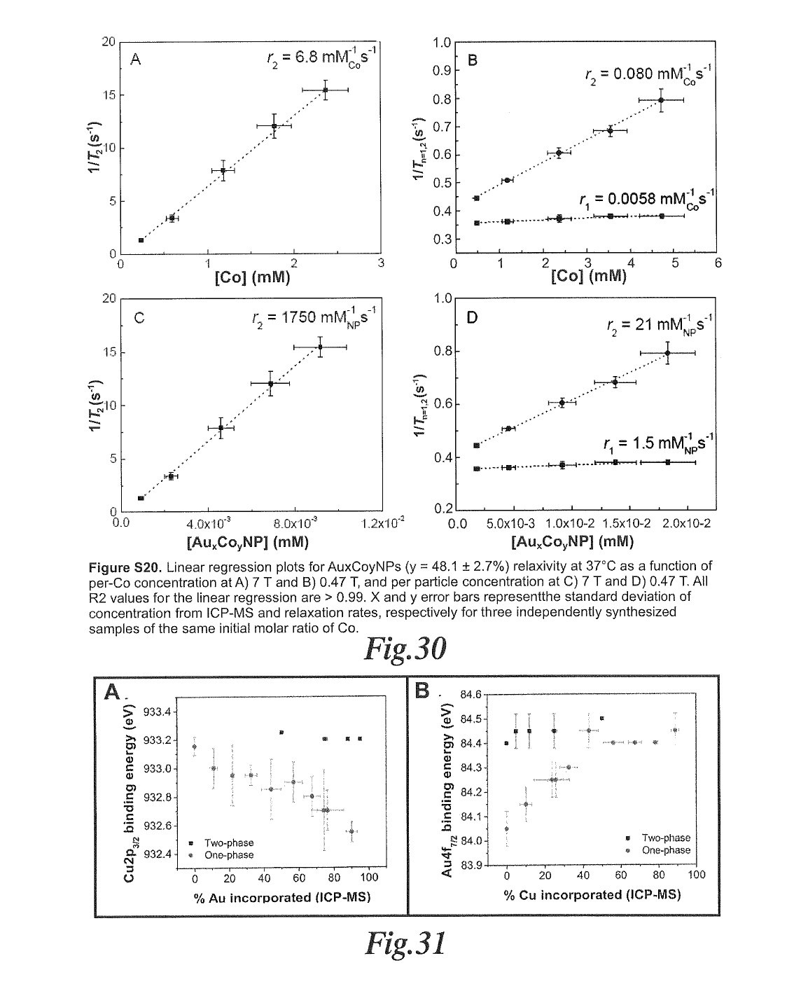

Example plots of relaxation rate vs. [Co] and [AuₓCo_{y}NPs] is shown in FIG. 30.

Although relaxivity values are commonly reported per metal concentration (mMₘₑₜₐₗ⁻¹s⁻¹), there is rationale to report per particle relaxivities when evaluating the relaxivity of superparamagnetic or paramagnetic nanoparticle systems. These systems typically act as T₂ agents via an outer sphere relaxation mechanism, in which the entire particle acts as a large paramagnetic ion per superparamagnetic phenomena. For the application as an MRI contrast agent, the figure of merit is the interaction of H₂O and a target (relaxation agent). Therefore, the per-particle relaxivity is reported to facilitate comparison in relaxivity values for particles of various sizes, compositions, and surface chemistry. The discrepancy between comparing per-metal values and per-particle values is highlighted in the discussion below. For the case of lone, chelated metal ions that act via an inner sphere mechanism to enhance proton relaxation rates, per-metal relaxivities are reported, due to the water interaction with a sole metal center. In order to compare our per-particle relaxivities to per-metal ion values reported in the literature, we converted all comparison values to per-particle relaxivities using the reported average diameter. For comparison to thoroughly studied iron oxide nanoparticles, a cubic spinel lattice structure was assumed for simplicity with a lattice constant=8.3 Å, as well as a AB₂O₄ formula, leading to 24 metal atoms per unit cell with no vacancies. The conversion from per-metal relaxivity to per-particle relaxivity is below in equation 9:

$${r}_{n = 1 , 2} ( {mM}_{{ }_{NP}}^{- 1} {s}^{- 1} ) = {r}_{n = 1 , 2} ( {mM}_{metal}^{- 1} {s}^{- 1} ) \cdot ( 24 metal atoms ) {( \frac{\frac{4}{3} π ( \frac{d}{2} )}{{lattice}^{3}} )}^{3} \qquad ( 9 )$$

Where d is particle diameter and lattice³ is the volume of the cubic unit cell.³

Table 7 includes both per-metal and per-particle relaxivity values at 0.47 T (r₁, r₂, and r₂/r₁) and 7 T (r₂) at 37° C. T₁ relaxivities are not reported at 7 T because even the most concentrated particles for each composition had a T₁ value equal to pure water, within error, reasonably so, given that as field strength increases, r₁ values decrease. 100% Au nanoparticles were measured as a control and did not show any linear correlation at either field strength.

We found that per-particle relaxivities for 100% CoNPs (d_{core}=2.9 nm, d_{H}=4.9 nm, r₂=12,200 mM_{NP}⁻¹s⁻¹, r₂=26 mM_{Co}⁻¹s⁻¹) were only an order of magnitude smaller when compared to the significantly larger (d_{core}=8.4 nm, d_{H}=300 nm), pharmaceutically available Ferumoxsil (r₂=937,877 mM_{NP}⁻¹s⁻¹, r₂=72 mM_{Fe}⁻¹s⁻¹), as well as the marketed as Ferumoxtran (d_{core}=4.9 nm, d_{H}=49.7 nm, r₂=137,038 mM_{NP}⁻¹s⁻¹, r₂=53 mM_{Fe}⁻¹s⁻¹) and Ferumoxide (d_{core}=4.8 nm, d_{H}=227 nm, r₂=260,066 mM_{NP}⁻¹s⁻¹, r₂=107 mM_{Fe}⁻¹s⁻¹).^{[17]} The 100% CoNPs exhibit T₂ relaxivities the same order of magnitude, though slightly smaller when compared to more recent PEG-capped Fe₃O₄ particles of comparable size (d_{core}=3 nm, r₂=27,890 mM_{NP}⁻¹s⁻¹, r₂=47 mM_{Fe}⁻¹s⁻¹).^{[18]} When compared to PEG-1000-coated iron oxide particles of similar diameter to AuₓCo_{y}NPs, (d_{core}=1.9 nm, r₂=2216 mM_{NP}⁻¹s⁻¹, r₂=14 mM_{Fe}⁻¹s⁻¹)^{[19]} the per-particle relaxivities for AuₓCo_{y}NPs (y=48.1±2.7% Co and y=62.0±2.0% Co) are comparable (r₂=1750 mM_{NP}⁻¹s⁻¹ (6.8 mM_{Co}⁻¹s⁻¹) and 3650 mM_{NP}⁻¹s⁻¹ (11 mM_{Co}⁻¹s⁻¹), respectively), while higher % Co compositions are larger by an order of magnitude. These properties make AuₓCo_{y}NPs suitable as negative contrast agents.

TABLE 7
Per-Co and per-particle relaxivity values measured at 0.47 T and 7 T at 37° C. for
select AuₓCo_{y}NPs compositions.

| | Per-Co relaxivity | | | | Per-particle relaxivity | | | |
|---|---|---|---|---|---|---|---|---|
| NP | | | | | | | | |
| composition | r₁ | r₂ | | r₂ | r₁ | r₂ | | r₂ |
| (% Co) | (mM_{Co}⁻¹s⁻¹) | (mM_{Co}⁻¹s⁻¹) | r₂/r₁ | (mM_{Co}⁻¹s⁻¹) | (mM_{NP}⁻¹s⁻¹) | (mM_{NP}⁻¹s⁻¹) | r₂/r₁ | (mM_{NP}⁻¹s⁻¹) |
| ICP-MS | 0.47 T | 0.47 T | 0.47 T | 7 T | 0.47 T | 0.47 T | 0.47 T | 7 T |
| 0 ± 0 | NA | NA | NA | NA | NA | NA | NA | NA |
| 7.7 ± 0.7 | 0.19 | 0.37 | 2.0 | 1.5 | 6.1 | 12 | 2.0 | 49 |
| 26.8 ± 1.9 | 0.041 | 0.16 | 4.0 | 2.4 | 3.6 | 14 | 4.0 | 209 |
| 48.1 ± 2.7 | 0.0058 | 0.080 | 14 | 6.8 | 1.5 | 21 | 4 | 1750 |
| 62.0 ± 2.0 | 0.0082 | 0.14 | 17 | 11 | 2.9 | 47 | 17 | 3650 |
| 100 ± 0 | 0.0083 | 0.23 | 27 | 26 | 4.0 | 109 | 7 | 12200 |

It should be understood that while this disclosure has been described herein in terms of specific embodiments set forth in detail, such embodiments are presented by way of illustration of the general principles of the disclosure, and the disclosure is not necessarily limited thereto. Certain modifications and variations in any given material, process step or chemical formula will be readily apparent to those skilled in the art without departing from the true spirit and scope of the present disclosure, and all such modifications and variations should be considered within the scope of the claims that follow.

What is claimed is:

1. A method for producing small nanoparticles of a discrete gold-cobalt nanoparticle alloy, comprising:
mixing, at room temperature in air, a first aqueous solution having a first molar ratio of HAuCl₄ and Co(NO₃)₂ with an organic ligand comprising poly(ethylene glycol) methyl ether thiol (PEGSH, average Mₙ=1000 Da) and a reducing agent comprising sodium borohydride (NaBH₄).

2. The method of claim 1 further comprising: characterizing the gold-cobalt nanoparticles of the first aqueous solution by photoluminescence, by other property of the gold-cobalt alloy nanoparticles from the first aqueous solution and/or by one or more of UV-visible spectroscopy, inductively coupled plasma mass spectrometry (ICP-MS), X-ray photoelectron spectroscopy (XPS), PL, HRTEM techniques, and ¹H nuclear magnetic resonance (NMR) techniques.

3. The method of claim 2 further comprising: mixing, at room temperature in air, a second aqueous solution having a second molar ratio of HAuCl₄ and Co(NO₃)₂ with an organic ligand comprising poly(ethylene glycol) methyl ether thiol (PEGSH, average Mₙ=1000 Da) and a reducing agent comprising sodium borohydride (NaBH₄).

4. The method of claim 3 further comprising: characterizing the gold-cobalt nanoparticles of the second aqueous solution by photoluminescence, by other property of the gold-cobalt alloy nanoparticles from the second aqueous solution and/or by one or more of UV-visible spectroscopy, inductively coupled plasma mass spectrometry (ICP-MS), X-ray photoelectron spectroscopy (XPS), PL, HRTEM techniques, and ¹H nuclear magnetic resonance (NMR) techniques.

5. The method of claim 4 further comprising: comparing the characterization results for the gold-cobalt nanoparticles from the first and second aqueous solutions.

6. A method for producing small nanoparticles of a discrete noble metal-transition metal nanoparticle alloy, comprising:
mixing, at room temperature in air, a first aqueous solution having a first molar ratio of a noble metal and a transition metal with an organic ligand and a reducing agent.

7. The method of claim 6 further comprising: characterizing the noble metal-transition metal nanoparticles of the first aqueous solution by photoluminescence, by other property of the noble metal-transition metal alloy nanoparticles from the first aqueous solution and/or by one or more of UV-visible spectroscopy, inductively coupled plasma mass spectrometry (ICP-MS), X-ray photoelectron spectroscopy (XPS), PL, HRTEM techniques, and 1H nuclear magnetic resonance (NMR) techniques.

8. The method of claim 7 further comprising: mixing, at room temperature in air, a second aqueous solution having a second molar ratio of noble metal-transition metal with an organic and a reducing agent.

9. The method of claim 8 further comprising: characterizing the noble metal-transition metal nanoparticles of the second aqueous solution by photoluminescence, by other property of the noble metal-transition metal alloy nanoparticles from the second aqueous solution and/or by one or more of UV-visible spectroscopy, inductively coupled plasma mass spectrometry (ICP-MS), X-ray photoelectron spectroscopy (XPS), PL, HRTEM techniques, and 1H nuclear magnetic resonance (NMR) techniques.

10. The method of claim 9 further comprising: comparing the characterization results for the noble metal-transition metal nanoparticles from the first and second aqueous solutions.

11. The method of claim 6 wherein the noble metal is selected from the group consisting of gold (Au), silver (Ag) and platinum (Pt) and the transition metal is selected from the group consisting of copper (Cu), cobalt (Co), nickel (Ni), zinc (Zn), ruthenium (Ru), rhodium (Rh), aluminum (Al), iron (Fe) and palladium (Pd).

12. A dual NIR-T₂-weighted contrast imaging agent comprising nanoparticles of a discrete gold-cobalt nanoparticle alloy having a composition of Co₈₀Au₂₀.

13. The dual NIR-T₂-weighted contrast imaging agent of claim 12 wherein an initial molar % Co=80% and an actual % Co incorporated=52%.

14. A dual NIR-T₂-weighted contrast imaging agent comprising nanoparticles of a discrete gold-cobalt nanoparticle alloy having a composition of Co₅₀Au₅₀.

15. The dual NIR-T₂-weighted contrast imaging agent of claim 14 wherein an initial molar % Co=15% and an actual % Co incorporated=62%.

16. The dual NIR-T₂-weighted contrast imaging agent of claim 12 wherein the gold-cobalt nanoparticles have a diameter ranging from about 2 nm to about 3 nm.

17. The dual NIR-T₂-weighted contrast imaging agent of claim 14 wherein the gold-cobalt nanoparticles have a diameter ranging from about 2 nm to about 3 nm.

18. The dual NIR-T₂-weighted contrast imaging agent of claim 16 wherein the gold-cobalt nanoparticles are capped with a biologically compatible capping ligand.

19. The dual NIR-T₂-weighted contrast imaging agent of claim 17 wherein the gold-cobalt nanoparticles are capped with a biologically compatible capping ligand.
